(19) United States
(12) Patent Application Publication
(10) Pub. No.: US 2005/0222088 A1
(43) Pub. Date: Oct. 6, 2005

(54) Synthesis of chiral furan amino acids as novel peptide building blocks

(76) Inventors: Tushar Kanti Chakraborty, Hyderabad (IN); Subhasish Tapadar, Hyderabad (IN)

Correspondence Address: Joseph J. Crimaldi;Renner, Otto, Boisselle & Sklar, LLP, Nineteenth Floor, 1621 Euclid Avenue, Cleveland, OH, 44115-2191, US

(21) Appl. No.: 10/814,525

(22) Filed: Mar. 31, 2004

(51) Int. Cl. A61K 31/695; A61K 31/665; A61K 31/4178; A61K 31/405

(52) U.S. Cl. 514/63; 514/471; 514/100; 514/397; 514/414; 548/465; 548/311.1; 549/484

(57) ABSTRACT

The present invention provides a chiral furan amino acids, in enantiomerically pure forms, either R or S. The starting materials are being used chiral N-terminal-protected amino aldehydes derived from the corresponding N-terminal-protected protected L- or D-amino acids. The present invention also relates to a process for preparing these chirally substituted furan amino acids constitute an important class of conformationally constrained peptide based molecules that can be used as dipeptide isosteres in peptidomimetic studies.

### FIELD OF INVENTION

The present invention relates to stereoselective chiral furan amino acids, an important class of peptide based molecules having a general structure as shown in 1 in Formula 1, and process for preparing the same. More Particularly, the novel chiral furan amino acids, carry a chiral center at the amino terminal with substituent resembling the side-chains of natural amino acids and stereoselective synthesis of these molecules in either R- or S-enantiomeric forms. The starting materials are being used chiral N-terminal-protected amino aldehydes derived from the corresponding N-terminal-protected protected L- or D-amino acids. 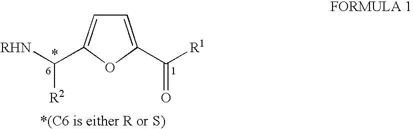
Wherein;
R═H, tert-butoxycarbonyl (Boc), benzyloxycarbonyl (Cbz), 9-fluorenylmethyl (Fmoc), acetyl or salts such as HCl.H, CF₃COOH.H and others;
R¹═—OH, —O-alkyl, —O-arylalkyl, -amine, -alkylamine, -arylalkylamine, and others
R²═CH₃—, (CH₃)₂CH—, (CH₃)₂CHCH₂—, CH₃CH₂CH(CH₃)—, alkyl groups, (OR³)CH₂—, CH₃(OR³)CH—, (R³S)CH₂—, CH₃SCH₂CH₂—, (RHN)CH₂CH₂CH₂CH₂—, (CONH₂)CH₂—, (CONH₂)CH₂CH₂—, (CO₂R⁴)CH₂—, (CO₂R⁴)CH₂CH₂—, Ph-, Ar—, PhCH₂—, ArCH₂—, Phenylalkyl-, arylalkyl-, (indolyl)CH₂—, (imidazolyl)CH₂—, and all other amino acid side-chains
R³═H, tert-butyl, alkyl, benzyl, arylCH₂, CO(alkyl), CO(arylalkyl), SO₃H, PO₃H₂, silyl and others
R⁴═H, tert-butyl, alkyl, benzyl, arylCH₂, and others
R—R²═—(CH₂)ₙ— (n=2, 3, 4 . . . )

### BACKGROUND OF THE INVENTION

In search of new molecular entities for discovering new drugs and materials, organic chemists are looking for innovative approaches that try to imitate nature in assembling quickly large number of distinct and diverse molecular structures from ‘nature-like’ and yet unnatural designer building blocks using combinatorial approach. This has become necessary today as it is being increasingly felt that natural products, or natural product based leads hold better promises for discovering new molecular entities as drugs (Rouhi, A. M. C&En 2003, 81(41), 77-91).¹ Peptide based molecules can play very important roles, in this aspect, in the development of new drugs. However, the use of peptides as drugs is limited by their low physiological stability in the gastrointestinal tract, loss of their original conformation once truncated from the native protein and their intrinsic flexibility because of which it is difficult to restrict short linear peptides in any particular conformation required to bind effectively to receptors. To overcome these problems, conformationally rigid non-peptide “scaffolds” can be inserted in the appropriate sites in the peptides to produce the specific secondary structure required for binding to the corresponding receptor. Compounds made of such unnatural building blocks are also expected to be more stable toward proteolytic cleavage in physiological systems than their natural counterparts. The unnatural building blocks developed for this purpose should be carefully designed to manifest the structural diversities of the monomeric units used by nature like amino acids, carbohydrates and nucleosides to build its arsenal.

In recent years, furan amino acid, 5-(aminomethyl)-2-furoic acid (Chakraborty, T. K. et al Tetrahedron Letters 2002, 43, 1317-1320)² and pyrrole amino acid, 5-(aminomethyl)-1H-pyrrole-2-carboxylic acid (Chakraborty, T. K. et al Tetrahedron Letters 2002, 43, 2589-2592; Chakraborty, T. K. et al Tetrahedron Letters 2003, 44, 471-473),³ have emerged as versatile templates that have been used as conformationally constrained scaffolds in peptidomimetic studies and as important class of synthetic monomers leading to de novo oligomeric libraries. These furan amino acid and pyrrole amino acid are designer building blocks bearing both amino and carboxyl functional groups on the regular furan and pyrrole frameworks, respectively, at C2 and C5 positions. There are several advantages of these building blocks. The rigid furan and pyrrole rings of these molecules make them ideal candidates as non-peptide scaffolds in peptidomimetics where they can be easily incorporated by using their carboxyl and amino termini utilizing well-developed solid-phase or solution-phase peptide synthesis methods. At the same time, it allows efficient exploitation of the structural constraints of these molecules to create the desired folded structures in small peptides required to bind to their receptors. The insertion of these scaffolds can also influence the hydrophobic/hydrophilic nature of the resulting peptidomimetic compounds.

Introduction of a chiral center in the amino terminus of these furan amino acids gives rise to an additional combinatorial site in these multifunctional building blocks that will not only help to induce desired secondary structure in peptides, but will also allow to mimic the side-chains of natural amino acids influencing the hydrophobicity/hydrophilicity of the resulting peptidomimetic molecules. While synthesis of unsubstituted 5-(aminomethyl)-2-furoic acid has been reported starting from fructose (Chakraborty, T. K. et al Tetrahedron Letters 2002, 43, 1317-1320),² introduction of a chiral center in its C6 position required a different approach.

Development of a robust synthetic strategy to construct these molecules in enantiomerically pure forms will allow their wide-ranging applications in peptidomimetic studies. The strategy adopted here allows synthesis of these molecules in either R- or S-enantiomeric forms depending on the chiralities of the starting amino acids.

The following abbreviation are used with the following meanings: CSA: camphor sulphonic acid; DMSO: dimethyl sulfoxide; PCC: pyridinium chlorochromate; Boc: tert-butoxycarbonyl; FmocOSu: 9-fluorenylmethyl N-succinimidyl carbonate; TFA: trifluoroacetic acid; DCC=N,N′-dicyclohexylcarbodiimide; HOBt=1-hydroxybenzotrazole.

Amino acids are denoted by L or D appearing before the symbol and separated from it by hyphen.

### OBJECTIVES OF THE INVENTION

The main objective of the invention is to provide stereoselective chiral furan amino acids, an important class of peptide based molecules having a general structure as shown in 1 in Formula 1. 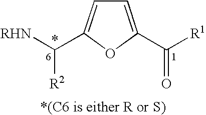
Wherein;
R═H, tert-butoxycarbonyl (Boc), benzyloxycarbonyl (Cbz), 9-fluorenylmethyl (Fmoc), acetyl or salts such as HCl.H, CF₃COOH.H and others;
R¹═—OH, —O-alkyl, —O-arylalkyl, -amine, -alkylamine, -arylalkylamine, and others
R²═CH₃—, (CH₃)₂CH—, (CH₃)₂CHCH₂—, CH₃CH₂CH(CH₃)—, alkyl groups, (OR³)CH₂—, CH₃(OR³)CH—, (R³S)CH₂—, CH₃SCH₂CH₂—, (RHN)CH₂CH₂CH₂CH₂—, (CONH₂)CH₂—, (CONH₂)CH₂CH₂—, (CO₂R⁴)CH₂—, (CO₂R⁴)CH₂CH₂—, Ph-, Ar—, PhCH₂—, ArCH₂—, Phenylalkyl-, arylalkyl-, (indolyl)CH₂—, (imidazolyl)CH₂—, and all other amino acid side-chains
R³═H, tert-butyl, alkyl, benzyl, arylCH₂, CO(alkyl), CO(arylalkyl), SO₃H, PO₃H₂, silyl and others
R⁴═H, tert-butyl, alkyl, benzyl, arylCH₂, and others
R—R²═—(CH₂)ₙ— (n=2, 3, 4 . . . ).

Another objective of the present invention is to provide a process for preparing novel chiral furan amino acids, carry a chiral center at the amino terminal with substituent resembling the side-chains of natural amino acids and stereoselective synthesis of these molecules in either R- or S-enantiomeric forms.

Yet another objective of the present invention is to provide novel furan amino acid peptide based molecules that carry a chiral center at the amino terminal, giving rise to an additional combinatorial site in these multifunctional molecules which can be used in various peptidomimetic studies to induce conformational constraints in small peptides.

### SUMMARY OF THE INVENTION

The present invention provides a chiral furan amino acids, in enantiomerically pure forms, either R or S. The starting materials are being used chiral N-terminal-protected amino aldehydes derived from the corresponding N-terminal-protected protected L- or D-amino acids. The present invention also relates to a process for preparing these chirally substituted furan amino acids constitute an important class of conformationally constrained peptide based molecules that can be used as dipeptide isosteres in peptidomimetic studies.

### DETAILED DESCRIPTION OF THE INVENTION

Accordingly the present invention provides an unnatural chiral furan amino acids carrying natural amino acid side-chains in C6-position and having a general structure 1 as shown in Formula 1. 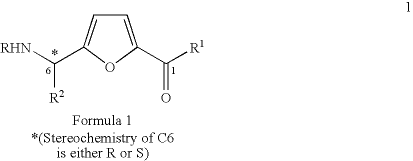
Wherein;
R═H, tert-butoxycarbonyl (Boc), benzyloxycarbonyl (Cbz), 9-fluorenylmethyl (Fmoc), acetyl or salts such as HCl, CF₃COOH.H and others;
R¹═—OH, —O-alkyl, —O-arylalkyl, -amine, -alkylamine, -arylalkylamine, and others;
R²═CH₃—, (CH₃)₂CH—, (CH₃)₂CHCH₂—, CH₃CH₂CH(CH₃)—, alkyl groups;
(OR³)CH₂—, CH₃(OR³)CH—, (R³S)CH₂—, CH₃SCH₂CH₂—, (RHN)CH₂CH₂CH₂CH₂—; (CONH₂)CH₂—, (CONH₂)CH₂CH₂—, (CO₂R⁴)CH₂—, (CO₂R⁴)CH₂CH₂—, Ph-, Ar—; PhCH₂—, ArCH₂—, Phenylalkyl-, arylalkyl-, (indolyl)CH₂—, (imidazolyl)CH₂—, and all other amino acid side-chains;
R³═H, tert-butyl, alkyl, benzyl, arylCH₂, CO(alkyl), CO(arylalkyl), SO₃H, PO₃H₂, silyl and others;
R⁴═H, tert-butyl, alkyl, benzyl, arylCH₂, and others;
R—R²═—(CH₂)ₙ— (n=2, 3, 4 . . . );

In an embodiment of the present invention, wherein if the stereochemistry of C6 is S and the substitutions are R¹=Me, R²=Me and R=Boc having a structural formula 2 shown here below

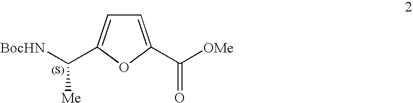

In another embodiment of the present invention, wherein if the stereochemistry of C6 is S and the substitutions are R¹═OH, R²=Me and R=Boc having a structural formula 3 shown here below

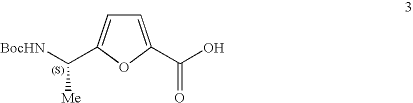

In yet another embodiment of the present invention, wherein if the stereochemistry of C6 is S and the substitutions are R¹=OMe, R²=Me and R═CF₃COOH.H having a structural formula 4 shown here below

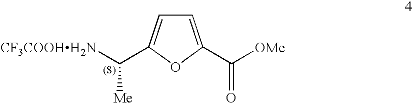

In yet another embodiment of the present invention, wherein if the stereochemistry of C6 is S and the substitutions are R¹═OH, R²=Me and R═CF₃COOH.H having a structural formula 5 shown here below

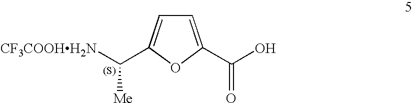

In still another embodiment of the present invention, wherein if the stereochemistry of C6 is R and the substitutions are R¹=OMe, R²=Me and R=Boc having a structural formula 6 shown here below

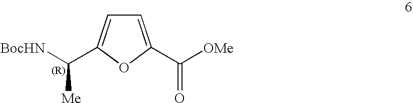

In a further embodiment of the present invention, wherein if the stereochemistry of C6 is R and the substitutions are R¹═OH, R²=Me and R=Boc having a structural formula 7 shown here below

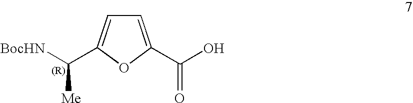

In one more embodiment of the present invention, wherein if the stereochemistry of C6 is R and the substitutions are R¹=OMe, R²=Me and R═CF₃COOH.H having a structural formula 8 shown here below

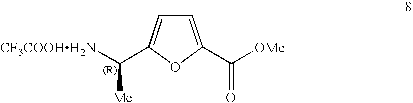

In one another embodiment of the present invention, wherein if the stereochemistry of C6 is R and the substitutions are R¹═OH, R²=Me and R═CF₃COOH.H having a structural formula 9 shown here below

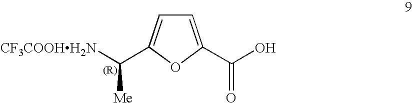

In another embodiment of the present invention, wherein if the stereochemistry of C6 is S and the substitutions are R¹=OMe, R²=CHMe₂ and R=Boc having a structural formula 10 shown here below

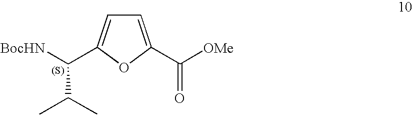

In yet another embodiment of the present invention, wherein if the stereochemistry of C6 is S and the substitutions are R¹═OH, R²=CHMe₂ and R=Boc having a structural formula 11 shown here below

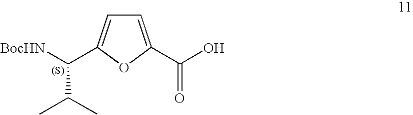

In yet another embodiment of the present invention, wherein if the stereochemistry of C6 is S and the substitutions are R¹=OMe, R²=CHMe₂ and R═CF₃COOH.H having a structural formula 12 shown here below

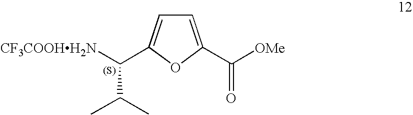

In one more embodiment of the present invention, wherein if the stereochemistry of C6 is S and the substitutions are R¹═OH, R²=CHMe₂ and R═CF₃COOH.H having a structural formula 13 shown here below

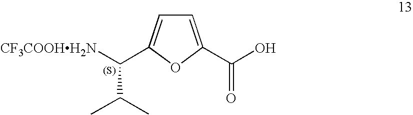

In one another embodiment of the present invention, wherein if the stereochemistry of C6 is R and the substitutions are R¹=OMe, R²=CHMe₂ and R=Boc having a structural formula 14 shown here below:

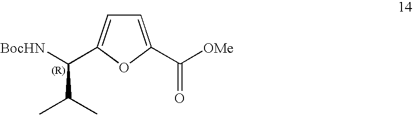

In still another embodiment of the present invention, wherein if the stereochemistry of C6 is R and the substitutions are R¹═OH, R²=CHMe₂ and R=Boc having a structural formula 15 shown here below

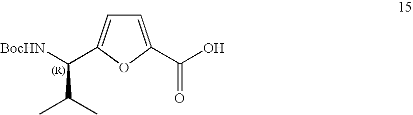

In yet another embodiment of the present invention, wherein if the stereochemistry of C6 is R and the substitutions are R¹=OMe, R²=CHMe₂ and R═CF₃COOH.H having a structural formula 16 shown here below

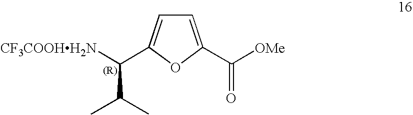

In yet another embodiment of the present invention, wherein if the stereochemistry of C6 is R and the substitutions are R¹═OH, R²=CHMe₂ and R═CF₃COOH.H having a structural formula 17 shown here below

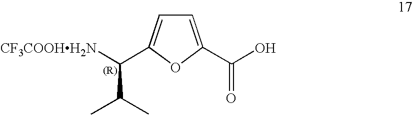

In a further embodiment of the present invention, wherein if the stereochemistry of C6 is S and the substitutions are R¹=OMe, R²=CH₂Ph and R=Boc having a structural formula 18 shown here below

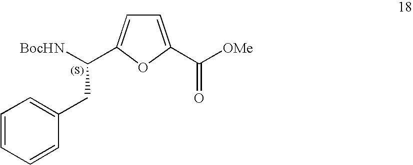

In a further more embodiment of the present invention, wherein if the stereochemistry of C6 is S and the substitutions are R¹═OH, R²=CH₂Ph and R=Boc having a structural formula 19 shown here below

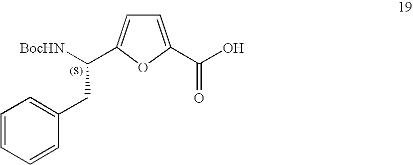

In one more embodiment of the present invention, wherein if the stereochemistry of C6 is S and the substitutions are R¹=OMe, R²=CH₂Ph and R═CF₃COOH.H having a structural formula 20 shown here below

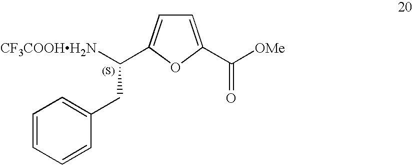

In another embodiment of the present invention, wherein if the stereochemistry of C6 is S and the substitutions are R¹═OH, R²=CH₂Ph and R═CF₃COOH.H having a structural formula 21 shown here below

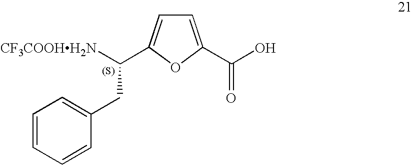

In yet another embodiment of the present invention, wherein if the stereochemistry of C6 is R and the substitutions are R¹=OMe, R²=CH₂Ph and R=Boc having a structural formula 22 shown here below

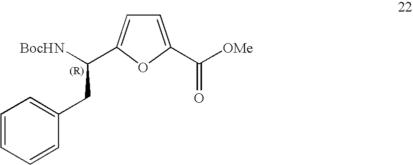

In yet another embodiment of the present invention, wherein if the stereochemistry of C6 is R and the substitutions are R¹═OH, R²=CH₂Ph and R=Boc having a structural formula 23 shown here below

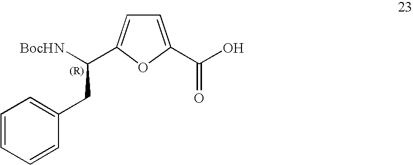

In yet another embodiment of the present invention, wherein if the stereochemistry of C6 is R and the substitutions are R¹=OMe, R²=CH₂Ph and R═CF₃COOH.H having a structural formula 24 shown here below

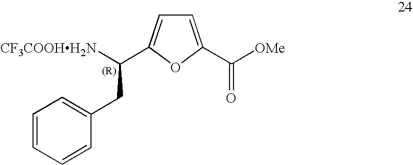

In a still another embodiment of the present invention, wherein if the stereochemistry of C6 is R and the substitutions are R¹═OH, R²=CH₂Ph and R═CF₃COOH.Hc having a structural formula 25 shown here below

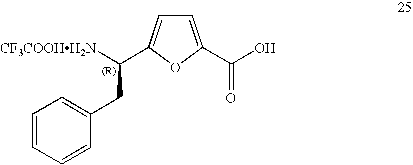

In yet another embodiment of the present invention, wherein N-Fmoc-protected furan amino acid is obtained by treatment with FmocOSu in dioxane-water in the ration of 1:1.

In still another embodiment of the present invention, wherein if structure 1 with substitution R=Boc, R¹═OH, R²=Me and 6S stereochemistry, has the following characteristics: R_{f}=0.45 (silica, 1:9 MeOH/CHCl₃ with 1% AcOH); [α]_{D}²³=−52.8 (c 1.14, MeOH); ¹H NMR (200 MHz, CDCl₃) δ 7.17 (br d, J=2.2 Hz, 1H, aromatic), 6.29 (d, J=2.2 Hz, 1H, aromatic), 5.04 (br m, 1H, NH), 4.93 (br m, 1H, CHNH), 1.48 (d, J=6.59 Hz, 3H, CH3), 1.42 (s, 9H, t-butyl) and yield up to 98%.

In one more embodiment of the present invention, wherein if structure 1 with substitution R=Boc, R¹═OH, R²=CHMe₂ and 6S stereochemistry, has the following characteristics: R_{f}=0.5 (silica, 1:9 MeOH/CHCl₃ with 1% AcOH); ¹H NMR (200 MHz, CDCl₃) δ 7.18 (br 1H, one of the furan ring protons), 6.39 (br, 1H, one of the furan ring protons), 5.09 (br, 1H, NH), 4.61 (br, 1H, CHNH), 2.2 (m, 1H, CH(CH₃)₂), 1.42 (s, 9H, t-butyl), 0.95 (d, J=6.69 Hz, 3H, CH₃), 0.89 (d, J=6.69 Hz, 3H, CH₃) and yield up to 88%.

In another embodiment of the present invention, wherein if structure 1 with substitution R=Boc, R¹═OH, R²=CH₂Ph and 6S stereochemistry, has the following characteristics: R_{f}=0.5 (silica, 10 MeOH/CHCl₃ with 1% AcOH); ¹H NMR (200 MHz, CDCl₃) δ 7.18 (m, 5H, aromatic protons), 7.05 (br, 1H, one of the furan ring protons), 6.12 (br, 1H, one of the furan ring protons), 5.03 (m, 2H, NH & CHNH), 3.16 (m, 2H, CH₂Ph), 1.39 (s, 9H, t-butyl) and yield up to 92%.

In yet another embodiment of the present invention, wherein if structure 1 with substitution R=Boc, R¹═OH, R²=Ph and 6S stereochemistry, has the following characteristics: R_{f}=0.5 (silica, 10% MeOH/CHCl₃ with 1% AcOH); ¹H NMR (200 MHz, CDCl₃) δ 7.29 (m, 5H, aromatic protons), 7.15 (br, 1H, one of the furan ring protons), 6.21 (br, 1H, one of the furan ring protons), 5.85 (br, 1H, CHNH), 5.43 (br, 1H, NH), 1.44 (s, 9H, t-butyl) and yield up to 90%.

In a further more embodiment of the present invention relates to a process for preparing unnatural chiral furan amino acids carrying natural amino acid side-chains in C6-position and having a general structure as shown in structure 1. 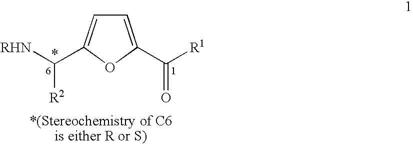
Wherein; R═H, Boc, Cbz, Fmoc, acetyl or salts such as HCl.H, CF₃COOH.H and others;
R¹═—OH, —O-alkyl, —O-arylalkyl, -amine, -alkylamine, -arylalkylamine, and others;
R²═CH₃—, (CH₃)₂CH—, (CH₃)₂CHCH₂—, CH₃CH₂CH(CH₃)—, alkyl groups; (OR³)CH₂—, CH₃(OR³)CH—, (R³S)CH₂—, CH₃SCH₂CH₂—, (RHN)CH₂CH₂CH₂CH₂—; (CONH₂)CH₂—, (CONH₂)CH₂CH₂—, (CO₂R⁴)CH₂—, (CO₂R⁴)CH₂CH₂—, Ph-, Ar—; PhCH₂—, ArCH₂—, Phenylalkyl-, arylalkyl-, (indolyl)CH₂—, (imidazolyl)CH₂—, and all other amino acid side-chains;
R³═H, tert-butyl, alkyl, benzyl, arylCH₂, CO(alkyl), CO(arylalkyl), SO₃H, PO₃H₂, silyl and others;
R⁴═H, tert-butyl, alkyl, benzyl, arylCH₂, and others;
R—R²═—(CH₂)— (n=2, 3, 4 . . . );
said process comprising the steps of:
   a) addition of Li-acetylide, prepared in-situ by reacting 3,4-O-isopropylidene-1,1-dibromobut-1-en-3,4-diol 3 with n-BuLi, to the chiral N-protected amino aldehyde 2 to obtain the propargyl alcohol adduct 4 as a mixture of isomers having the structure 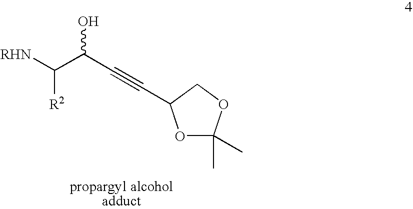
   b) selective hydrogenation of the acetylenic moiety to a cis double bond using P2-Ni to get the cis-allylic alcohol intermediate 5 having the structure 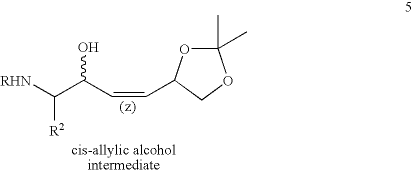
   c) treating 5 with acid to deprotect the acetonide and to furnish an intermediate triol
   d) selective acylation of the primary hydroxyl group of the triol from of step (c) to obtain the “cis-2-butene-1,4-diol” intermediate 6 having the structure 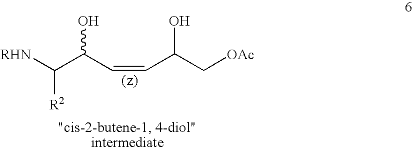
   e) oxidation of the “cis-2-butene-1,4-diol” intermediate 6 using pyridinium chlorochromate (PCC) to construct the furan ring
   f) deprotection of the intermediate acetate from step (e) in presence of anhydrous K₂CO₃ to obtain the chiral furanyl alcohol intermediate 7 having the structure 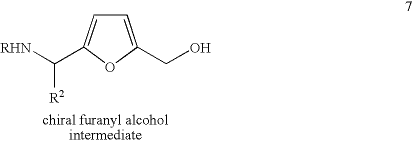
   g) oxidation of the primary hydroxyl of the chiral furanyl alcohol intermediate 7 using Swern oxidation process or SO₃-py complex to obtain an aldehyde
   h) further oxidation of the aldehyde intermediate from step (g) using NaClO₂—H₂O₂ to obtain the desired acid 1 (R¹ ═OH) having the structure 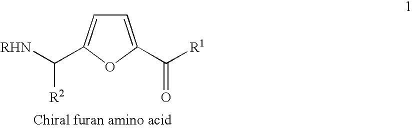
   i) transformation of the acid from step (h) into (a) an ester (i) on treatment with CH₂N₂ in ether (1: R¹=OMe), or (ii) an alcohol in the presence of acid (1: R¹═O-alkyl etc.); (b) an amide on treatment with an amine in presence of DCC and HOBt (1: R¹=-amine, -alkylamine, -arylalkylamine).

In an embodiment of the present invention, wherein if structure 4 with substitution R=Boc, R²=Me and 6S stereochemistry, has the following characteristics: R_{f}=0.5 (silica, 2:3 ethyl acetate/hexane); ¹H NMR (300 MHz, CDCl₃) δ 4.73-4.68 (ddd, J=6.04, 3.78, 1.51 Hz, 1H, CHOH), 4.65-4.62 (d, J=8.31 Hz, 1H, NH), 4.36-4.32 (ddd, J=6.79, 5.29, 1.51 Hz, 1H, CHCH₂), 4.15-4.09 (dd, J=6.79, 6.04 Hz, 1H, one of the CH₂ protons), 3.91-3.86 (dd, J=6.04, 5.29 Hz, 1H, one of the CH₂ protons), 3.83-3.76 (m, 1H, CHNH), 2.89 (bs, 1H, OH), 1.45 (s, 3H, acetonide methyl protons), 1.442 (s, 9H, t-butyl protons), 1.354 (s, 3H, acetonide methyl protons), 1.247-1.225 (d, J=6.79 Hz, 3H, CH₃) and yield up to 60%.

In another embodiment of the present invention, wherein structure 4 with substitution R=Boc, R²=CHMe₂ and 6S stereochemistry, has the following characteristics: R_{f}=0.5 (silica, 40% EtOAc/Hexane); ¹H NMR (300 MHz, CDCl₃) δ 4.7 (m, 1H, CHOH), 4.59 (d, J=9.07 Hz, 1H, NH), 4.12 (m, 1H, CHCH₂), 3.88 (m, 2H, CH₂),3.54 (m, 1H, CHNH), 1.78 (m, 1H, CH(CH₃)₂), 1.46 (s, 9H, t-butyl), 1.45 (s, 6H, acetonide protons), 0.99 (d, J=6.8 Hz, 6H, CH₃) and yield up to 63%.

In one more embodiment of the present invention, wherein if structure 4 with substitution R=Boc, R²=CH₂Ph and 6S stereochemistry, has the following characteristics: R_{f}=0.45 (silica, 40% EtOAc/Hexane); ¹H NMR (200 MHz, CDCl₃) δ 7.23 (m, 5H, aromatic protons), 4.82-4.65 (m, 2H, CHOH & NH), 4.37 (br, 1H, CHNH), 4.19-4.06 (m, 2H, CH & one of the CH₂), 3.9 (m, 1H, one of the CH₂), 2.91 (m, 2H, CH₂Ph), 1.39-1.38 (m, 15H, t-butyl & acetonide methyls) and yield up to 65%.

In another embodiment of the present invention, wherein if structure 4 with substitution R=Boc, R²=Ph and 6S stereochemistry, has the following characteristics: R_{f}=0.45 (silica, 40% EtOAc/Hexane); ¹H NMR (200 MHz, CDCl₃) δ 7.29 (m, 5H, aromatic protons), 5.27-5.18 (m, 2H, CHOH & NH), 5 (m, 1H, CHNH), 4.94 (m, 1H, CH), 4.03 (m, 2H, CH₂), 1.44 (s, 9H, t-butyl), 1.41 (s, 6H, acetonide methyls) and yield up to 62%.

In yet another embodiment of the present invention, wherein if structure 5 with substitution R=Boc, R²=Me and 6S stereochemistry, has the following characteristics: R_{f}=0.45 (silica, 2:3 ethyl acetate/hexane); ¹H NMR (200 MHz, CDCl₃) δ 5.62-5.55 (m, 2H, olefinic protons), 4.92-4.68 (m, 2H, CHOH), 4.36-4.27 (bs, 1H, NH), 4.15-4.05 (m, 2H, CH₂OH), 3.71-3.61 (m, 1H, CH), 3.06 (bs, 1H, OH), 1.44 (s, 9H, t-butyl protons), 1.40 (s, 3H, acetonide methyl protons), 1.36 (s, 3H, acetonide methyl protons), 1.18-1.15 (d, J=6.69 Hz, 3H, methyl protons) and yield up to 70%.

In yet another embodiment of the present invention, wherein if structure 5 with substitution R=Boc, R²=CHMe₂ and 6S stereochemistry, has the following characteristics: R_{f}=0.45 (silica, 30% EtOAc/Hexane); ¹H NMR (300 MHz, CDCl₃) δ 5.65 (m, 1H, olefinic proton), 5.54 (m, 1H, olefinic proton), 4.71 (bs, 1H, NH), 4.5 (m, 1H, CHOH), 4.09 (m, 1H, CH), 3.55 (m, 2H, CH₂), 3.24 (m, 1H, CHNH), 1.94 (m, 1H, CH(CH₃)₂), 1.44 (s, 9H, t-butyl), 1.43 (s, 6H, acetonide methyls), 1.0 (d, J=6.8 Hz, 3H, CH₃), 0.93 (d, J=6.8 Hz, 3H, CH₃) and yield up to 60%.

In yet another embodiment of the present invention, wherein if structure 5 with substitution R=Boc, R²=CH₂Ph and 6S stereochemistry, has the following characteristics: R_{f}=0.45 (silica, 40% EtOAc/Hexane); ¹H NMR (200 MHz, CDCl₃) δ 7.21 (m, 5H, aromatic protons), 5.82-5.55 (m, 2H, olefinic protins), 4.78 (m, 1H, NH), 4.62-4.34 (m, 2H, CHOH & CH), 4.06 (m, 1H, CHNH), 3.51 (m, 2H, CH₂), 2.85 (m, 2H, CH₂Ph), 1.39-1.32 (m, 15H, t-butyl & acetonide methyls) and yield up to 65%.

In yet another embodiment of the present invention, wherein if structure 5 with substitution R=Boc, R²=Ph and 6S stereochemistry, has the following characteristics: R_{f}=0.45 (silica, 40% EtOAc/hexane); ¹H NMR (200 MHz, CDCl₃) δ 7.25 (m, 5H, aromatic protons), 5.87-5.55 (m, 2H, olefinic protons), 5.25 (m, 2H, CHOH, NH), 4.99 (m, 1H, CHNH), 4.58 (m, 1H, CH), 3.90 (m, 2H, CH₂), 1.44 (s, 9H, t-butyl), 1.41 (s, 6H, acetonide methyls) and yield up to 70%.

In still another embodiment of the present invention, wherein if structure 6 with substitution R=Boc, R²=Me and 6S stereochemistry, has the following characteristics: R_{f}=0.6 (silica, 1:9 methanol/chloroform); ¹H NMR (200 MHz, CDCl₃) δ 5.66-5.46 (two dd, J=11.89, 6.69 Hz, 2H, olefinic protons), 4.90-4.85 (d, J=8.92 Hz, 1H, NH), 4.66-4.59 (dt, J=6.69, 4.46 Hz, 1H, CHOH), 4.41-4.36 (ddd, J=6.69, 5.02, 4.46 Hz, 1H, CHOH), 4.16-3.98 (two dd, J=11.15, 6.69 and 11.15, 4.46 Hz, 2H, CH₂OAc), 2.09 (s, 3H, CH₃CO), 1.44 (s, 9H, t-butyl), 1.20-1.17 (d, J=6.69 Hz, 3H, CH₃) and yield up to 93%.

In still one more embodiment of the present invention, wherein if structure 6 with substitution R=Boc, R²=CHMe₂ and 6S stereochemistry, has the following characteristics: R_{f}=0.45 (silica, 10% MeOH/CHCl₃); ¹H NMR (300 MHz, CDCl₃) δ 5.66 (dd, J=11.33, 7.93 Hz, 1H, olefinic proton), 5.54 (dd, J=11.33, 8.31 Hz, 1H, olefinic proton), 4.72-4.67 (m, 1H, CHOH), 4.4 (dd, J=7.93, 6.8 Hz, 1H, CH), 4.18 (dd, J=11.33, 3.4 Hz, 1H one of the CH₂), 3.93 (dd, J=11.33, 7.55 Hz, 1H, one of the CH₂), 2.1 (s, 3H, COCH₃), 2 (m, 1H, CH(CH₃)₂), 1.42 (s, 9H, t-butyl), 0.97 (d, J=6.8 Hz, 3H, CH₃), 0.92 (d, J=6.8 Hz, 3H, CH₃) and yield up to 80%.

In yet another embodiment of the present invention, wherein if structure 6 with substitution R=Boc, R²=CH₂Ph and 6S stereochemistry, has the following characteristics: R_{f}=0.45 (silica, 10% MeOH/CHCl₃); ¹H NMR (200 MHz, CDCl₃) δ 7.21 (m, 5H, aromatic protons), 5.68-5.45 (m, 2H, olefinic protons), 4.65 (m, 2H, CHOH & NH), 4.45 (m, 1H, CHOH), 4.05 (m, 2H, CH₂), 3.8 (m, 1H, CHNH), 2.85 (m, 2H, CH₂Ph), 2.04 (s, 3H, COCH₃), 1.25 (m, 15H, t-butyl) and yield up to 90%.

In yet another embodiment of the present invention, wherein if structure 6 with substitution R=Boc, R²=Ph and 6S stereochemistry, has the following characteristics: R_{f}=0.45 (silica, 10% MeOH/CHCl₃); ¹H NMR (200 MHz, CDCl₃) δ 7.29 (m, 5H, aromatic protons), 5.87-5.55 (m, 2H, olefinic protons), 5.25 (m, 2H, CHOH & NH), 4.85 (m, 1H, CHNH), 4.61 (m, 1H, CHOH), 4.21 (m, 2H, CH₂), 2.1 (s, 3H, COCH₃), 1.44 (s, 9H, t-butyl) and yield up to 85%.

In a further embodiment of the present invention, wherein if structure 7 with substitution R=Boc, R²=Me and 6S stereochemistry, has the following characteristics: R_{f}=0.45 (silica, 1:1 ethyl acetate/hexane); [α]_{D}²³=−59.9 (c 1.76, CHCl₃); ¹H NMR (200 MHz, CDCl₃) δ 6.17-6.14 (d, J=2.97 Hz, 1H, one of the ring protons), 6.08-6.04 (d, J=2.97 Hz, 1H, one of the ring protons), 4.86-4.71 (bs, 2H, NH and CH), 4.52 (s, 2H, CH₂OH), 2.14-1.93 (bs, 1H, OH) 1.48-1.43 (s, 12H, t-butyl group and methyl protons) and yield up to 98%.

In a further more embodiment of the present invention, wherein if structure 7 with substitution R=Boc, R²=CHMe₂ and 6S stereochemistry, has the following characteristics: R_{f}=0.5 (silica, 30% EtOAc/Hexane); [α]_{D}²³=−59.9 (c 1.76, CHCl₃); ¹H NMR (300 MHz, CDCl₃) δ 6.16 (d, J=2.93 Hz, 1H, one of the furan ring protons), 6.06 (d, J=2.93 Hz, 1H, one of the furan ring protons), 4.84 (d, J=8.79 Hz, 1H, NH), 4.53 (s, 2H, CH₂OH), 4.52 (m, 1H, CHNH) 2.09 (m, 1H, CH(CH₃)₂), 1.44 (s, 9H, t-butyl), 0.94 (d, J=6.59 Hz, 3H, CH₃), 0.88 (d, J=6.59 Hz, 3H, CH₃) and yield up to 95%.

In yet another embodiment of the present invention, wherein if structure 7 with substitution R=Boc, R²=CH₂Ph and 6S stereochemistry, has the following characteristics: R_{f}=0.5 (silica, 40% EtOAc/hexane); ¹H NMR (200 MHz, CDCl₃) δ 7.2 (m, 3H, aromatic protons), 7.02 (m, 2H, aromatic protons), 6.12 (d, J=2.97 Hz, 1H, one of the furan ring protons), 5.93 (d, J=2.97 Hz, 1H, one of the furan ring protons), 4.94 (m, 1H, CHNH), 4.81 (d, J=8.92 Hz, 1H, NH), 4.53 (s, 2H, CH₂OH), 3.09 (d, J=6.69 Hz, 2H, CH₂Ph), 1.39 (s, 9H, t-butyl) and yield up to 96%.

In still another embodiment of the present invention, wherein if structure 7 with substitution R=Boc, R²=Ph and 6S stereochemistry, has the following characteristics: R_{f}=0.45 (silica, 40% EtOAc/Hexane); ¹H NMR (400 MHz, CDCl₃) δ 7.29 (m, 5H, aromatic protons), 6.16 (d, J=3.05 Hz, 1H, one of the furan ring protons), 6.02 (d, J=3.05 Hz, 1H, one of the furan ring protons), 5.87 (br, 1H, NH), 5.25 (d, J=8.52 Hz, 1H, CHNH), 4.51 (s, 2H, CH₂OH), 1.44 (s, 9H, t-butyl) and yield up to 95%.

The present invention relates to the stereoselective construction of chiral furan amino acids, an important class of peptide building blocks, having a general structure as shown in 1 in Formula 1, in 8 steps (9 steps, for ester or amide) (Scheme 1) using chiral N-terminal-protected amino aldehydes as starting materials that could also be derived from the corresponding N-terminal-protected protected L- or D-amino acids, like for example, Boc-L-Ala-OH, Boc-D-Ala-OH, Boc-L-Phe-OH, Boc-D-Phe-OH, Boc-L-Val-OH, Boc-L-Val-OH, Boc-L-Leu-OH, Boc-L-Leu-OH, Boc-L-Ile-OH, Boc-D-Ile-OH, Boc-L-Ser(Bzl)-OH, Boc-D-Ser(Bzl)-OH, Boc-L-Thr(Bzl)-OH, Boc-D-Thr(Bzl)-OH, Boc-L-Tyr(Bzl)-OH, Boc-D-Tyr(Bzl)-OH, Fmoc-L-Ala-OH, Fmoc-D-Ala-OH, Fmoc-L-Phe-OH, Fmoc-D-Phe-OH, Fmoc-L-Val-OH, Fmoc-L-Val-OH, Fmocc-L-Leu-OH, Fmoc-L-Leu-OH, Fmoc-L-Ile-OH, Fmoc-D-Ile-OH, Fmoc-L-Ser(But)-OH, Boc-D-Ser(But)-OH, Fmoc-L-Thr(But)-OH, Fmoc-D-Thr(But)-OH, Fmoc-L-Tyr(But)-OH, Fmoc-D-Tyr(But)-OH and other appropriately protected amino acids, by converting them first to Weinreb amide, followed by reduction to aldehyde using LiAlH₄ (Fehrentz, J.-A. et al Synthesis 1983, 676-678).⁴ 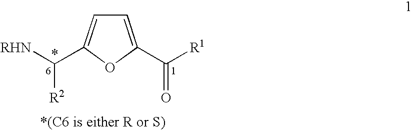
wherein;
R═H, tert-butoxycarbonyl (Boc), benzyloxycarbonyl (Cbz), 9-fluorenylmethyl (Fmoc), acetyl or salts such as HCl.H, CF₃COOH.H and others;
R¹═—OH, —O-alkyl, —O-arylalkyl, -amine, -alkylamine, -arylalkylamine, and others
R²═CH₃—, (CH₃)₂CH—, (CH₃)₂CHCH₂—, CH₃CH₂CH(CH₃)—, alkyl groups, (OR³)CH₂—, CH₃(OR³)CH—, (R³S)CH₂—, CH₃SCH₂CH₂—, (RHN)CH₂CH₂CH₂CH₂—, (CONH₂)CH₂—, (CONH₂)CH₂CH₂—, (CO₂R⁴)CH₂—, (CO₂R⁴)CH₂CH₂—, Ph-, Ar—, PhCH₂—, ArCH₂—, Phenylalkyl-, arylalkyl-, (indolyl)CH₂—, (imidazolyl)CH₂—, and all other amino acid side-chains
R³═H, tert-butyl, alkyl, benzyl, arylCH₂, CO(alkyl), CO(arylalkyl), SO₃H, PO₃H₂, silyl and others
R⁴═H, tert-butyl, alkyl, benzyl, arylCH₂, and others
R—R²═—(CH₂)ₙ— (n=2, 3, 4 . . . )

### Formula 1

Synthesis of Chiral Furan Amino Acids

The synthetic protocol developed in the present invention for the stereoselective synthesis of C6-substituted furan amino acids, 1 in Formula 1, may suitably be employed to synthesize any of the two enantiomers, R or S, in optically pure form. The details of the synthesis involving 8 steps (9 steps, for ester or amide) are shown in Scheme 1. Treatment of chiral N-protected amino aldehyde 2 derived from the corresponding amino acid (Reetz, M. T. et al Org. Synth. 1998, 76, 110; Reetz, M. T. Chem. Rev. 1999, 99, 1121-1162)⁵ with the Li-acetylide prepared in-situ by reacting 3,4-O-isopropylidene-1,1-dibromobut-1-en-3,4-diol 3 (Gung, B. W. et al J. Org. Chem. 2003, 68, 5956-5960)⁶ with n-BuLi, gave the propargyl alcohol adduct 4 as a mixture of isomers. Cis-hydrogenation of 4 using P2-Ni (Brown, C. A. et al J. Chem. Soc., Chem. Commun. 1973, 553; Brown, C. A. et al J. Org. Chem. 1973, 38, 2226)⁷ provided the cis-allylic alcohol intermediate 5. Treatment of 5 with acid led to the deprotection of the acetonide and the primary hydroxyl was selectively protected as acetate to get the “cis-2-butene-1,4-diol” intermediate 6. The resulting “cis-2-butene-1,4-diol” moiety of 6 was next transformed into a furan ring on oxidation with pyridinium chlorochromate (PCC) (Nishiyama, H. et al Chemistry Lett. 1981, 1363-1366)⁸ which was followed by the treatment of the intermediate with anhydrous K₂CO₃ to deprotect the acetate to give the chiral furanyl alcohol intermediate 7. Finally, a two-step oxidation process, (i) Swern oxidation or oxidation by SO₃-py complex to aldehyde, and (ii) oxidation of the aldehyde to acid using NaClO₂—H₂O₂, converted the primary hydroxyl group of 7 into the acid functionality (1: R¹═OH), which was transformed into (a) an ester (i) on treatment with CH₂N₂ in ether (1: R¹=OMe), or (ii) an alcohol in presence of acid (1: R¹═O-alkyl etc.); (b) an amide on treatment with an amine in presence of DCC and HOBt (1: R¹=-amine, -alkylamine, -arylalkylamine).

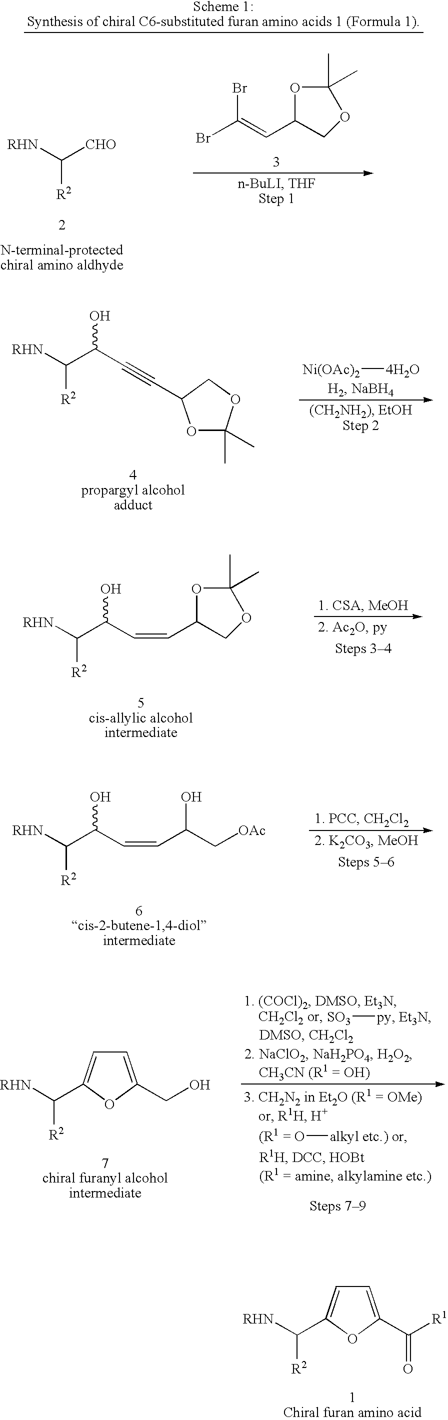

### EXAMPLE 1

### Process for Preparing Chiral Furan Amino Acid 1 Wherein C6 Stereochemistry is S and the Substitutions are R=Boc, R¹═OH, R²=Me

Step 1: Preparation of the Propargyl Alcohol Adduct 4 (R=Boc, R²=Me with 6S Stereochemistry)

To a solution of the dibromo compound 3 (7.82 g) in THF (110 mL) at −78° C., nBuLi (1.6 M in hexane, 32.5 mL) was slowly added with stirring. Stirring was continued at −78° C. for 30 minutes and then at room temperature for another 30 minutes, recooled to −78° C. and the aldehyde N-Boc-L-alaninal (2: R=Boc, R²=Me with 6S stereochemistry) (4.0 g), dissolved in THF (20 mL), was added. After 30 minutes, the reaction mixture was quenched with saturated aqueous NH₄Cl solution. The organic layer was separated and the aqueous layer was extracted with ethyl acetate. The combined organic layer was washed with brine and dried over anhydrous Na₂SO₄. The solvents were removed in rotary evaporator and the crude mixture was purified using flash column chromatography to afford the propargyl alcohol adduct 4 (R=Boc, R²=Me with 6S stereochemistry) (4.12 g) as oil in 60% yield. Data for 4 (R=Boc, R²=Me with 6S stereochemistry): R_{f}=0.5 (silica, 2:3 ethyl acetate/hexane); ¹H NMR (300 MHz, CDCl₃) δ 4.73-4.68 (ddd, J=6.04, 3.78, 1.51 Hz, 1H, CHOH), 4.65-4.62 (d, J=8.31 Hz, 1H, NH), 4.36-4.32 (ddd, J=6.79, 5.29, 1.51 Hz, 1H, CHCH₂), 4.15-4.09 (dd, J=6.79, 6.04 Hz, 1H, one of the CH₂ protons), 3.91-3.86 (dd, J=6.04, 5.29 Hz, 1H, one of the CH₂ protons), 3.83-3.76 (m, 1H, CHNH), 2.89 (bs, 1H, OH), 1.45 (s, 3H, acetonide methyl protons), 1.442 (s, 9H, t-butyl protons), 1.354 (s, 3H, acetonide methyl protons), 1.247-1.225 (d, J=6.79 Hz, 3H, CH₃).

Step 2: Preparation of the Cis-Allylic Alcohol Intermediate 5 (R=Boc, R²=Me with 6S Stereochemistry)

Nickel acetate tetrahydrate (2.5 g) was dissolved in 95% ethanol (110 mL) and placed under H₂. A solution of NaBH₄ in absolute ethanol (1 M, 10 mL) was added to it under room temperature, followed after 30 minutes by ethylene diamine (2.67 mL) and compound 4 (3.0 g) dissolved in ethanol. The reaction was monitored by TLC. Upon completion, it was diluted by addition of diethyl ether and filtered through Celite pad. The organic extract was washed with brine, dried (Na₂SO₄) and concentrated. Flash chromatography of the residue afforded pure cis-allylic alcohol intermediate 5 (R=Boc, R²=Me with 6S stereochemistry) (2.1 g, 70% yield) as colorless oil. Data for 5 (R=Boc, R²=Me with 6S stereochemistry): R_{f}=0.45 (silica, 2:3 ethyl acetatelhexane); ¹H NMR (200 MHz, CDCl₃) δ 5.62-5.55 (m, 2H, olefinic protons), 4.92-4.68 (m, 2H, CHOH), 4.36-4.27 (bs, 1H, NH), 4.15-4.05 (m, 2H, CH₂OH), 3.71-3.61 (m, 1H, CH), 3.06 (bs, 1H, OH), 1.44 (s, 9H, t-butyl protons), 1.40 (s, 3H, acetonide methyl protons), 1.36 (s, 3H, acetonide methyl protons), 1.18-1.15 (d, J=6.69 Hz, 3H, methyl protons).

Steps 3-4: Preparation of the “cis-2-butene-1,4-diol” Intermediate 6 (1R=Boc, R²=Me with 6S Stereochemistry)

A solution of compound 5 (R=Boc, R²=Me with 6S stereochemistry) (1.5 g) in methanol (20 mL) was treated with CSA (1.15 g) at 0° C. After 4 h, the reaction was quenched by adding saturated aqueous NaHCO₃ solution (till pH 8) and extracted with ethyl acetate. The combined organic extracts were washed with brine, dried (Na₂SO₄) and concentrated. The crude mixture was purified by flash chromatography to afford the triol (914 mg, 70% yield).

To a solution of the triol (0.843 g) in CH₂Cl₂ (15 mL) at −78° C. were added 2,4,6-collidine (0.85 mL) followed by acetyl chloride (0.25 mL). After 8 h, it was quenched by adding saturated aqueous NH₄Cl solution, extracted with ethyl acetate, washed with 1N HCl, water, brine, dried and concentrated. Column chromatography of the residue afforded pure mono acetylated “cis-2-butene-1,4-diol” intermediate 6 (R=Boc, R²=Me with 6S stereochemistry) (910 mg, 93% yield) as colorless oil. Data for 6 (R=Boc, R²=Me with 6S stereochemistry): R_{f}=0.6 (silica, 1:9 methanol/chloroform); ¹H NMR (200 MHz, CDCl₃) δ 5.66-5.46 (two dd, J=11.89, 6.69 Hz, 2H, olefinic protons), 4.90-4.85 (d, J=8.92 Hz, 1H, NH), 4.66-4.59 (dt, J=6.69, 4.46 Hz, 1H, CHOH), 4.41-4.36 (ddd, J=6.69, 5.02, 4.46 Hz, 1H, CHOH), 4.16-3.98 (two dd, J=11.15, 6.69 and 11.15, 4.46 Hz, 2H, CH₂OAc), 2.09 (s, 3H, CH₃CO), 1.44 (s, 9H, t-butyl), 1.20-1.17 (d, J=6.69 Hz, 3H, CH₃).

Steps 5-6: Preparation of the Chiral Furanyl Alcohol Intermediate 7 (R=Boc, R²=Me with 6S Stereochemistry)

To a solution of compound 6 (R=Boc, R²=Me with 6S stereochemistry) (0.8 g) in CH₂Cl₂ (30 mL), pyridinium chlorochromate (PCC, 1.02 g) was added. After 30 minutes, the reaction mixture was diluted with excess diethyl ether. The organic layer was washed with 1N HCl, water, brine and dried (Na₂SO₄). After concentration, the residual oil was purified by column chromatography to give pure 2,5-disubstituted furan derivative (0.337 g, 45% yield) as colorless oil.

The resulting furan (315 mg) was dissolved in methanol (5 mL), cooled to 0° C., and then anhydrous potassium carbonate (306 mg) was added. The reaction mixture was stirred at the same temperature for 15 minutes. It was diluted with ethyl acetate and washed with water, brine, dried (Na₂SO₄) and concentrated. Purification by column chromatography afforded the chiral furanyl alcohol intermediate 7 (R=Boc, R²=Me with 6S stereochemistry) (266 mg, 98% yield) as colorless oil. Data for 7 (R=Boc, R²=Me with 6S stereochemistry): R_{f}=0.45 (silica, 1:1 ethyl acetate/hexane); [α]_{D}²³=−59.9 (c 1.76, CHCl₃); ¹H NMR (200 MHz, CDCl₃) δ 6.17-6.14 (d, J=2.97 Hz, 1H, one of the ring protons), 6.08-6.04 (d, J=2.97 Hz, 1H, one of the ring protons), 4.86-4.71 (bs, 2H, NH and CH), 4.52 (s, 2H, CH₂OH), 2.14-1.93 (bs, 1H, OH) 1.48-1.43 (s,12H, t-butyl group and methyl protons).

Steps 7-8: Preparation of the Chiral Furan Amino Acid 1 (R=Boc, R¹═OH, R²=Me with 6S Stereochemistry)

Compound 7 (R=Boc, R²=Me with 6S stereochemistry) (260 mg) was oxidized to aldehyde in 80% yield by standard Swern oxidation procedure. A solution of oxalyl chloride (1.5 molar equiv) in dry CH₂Cl₂, cooled to −78° C., was treated with DMSO (3.0 molar equiv). After 5 min, the alcohol 7 (R=Boc, R²=Me with 6S stereochemistry) (1.0 molar equiv) dissolved in CH₂Cl₂ was added to the reaction mixture at the same temperature. After stirring for 1 h at −78° C., the reaction mixture was treated with Et₃N (5.0 molar equiv), slowly warmed to 0° C., and stirred at this temperature for 15 min. It was then poured into a cold saturated aqueous NH₄Cl solution and extracted with EtOAc. The combined organic extracts were washed with brine, dried (Na₂SO₄), filtered, and concentrated in vacuo. Purification by column chromatography afforded the aldehyde intermediate (206 mg, 80% yield) as oil.

To a solution of the aldehyde (190 mg) in CH₃CN (4 mL) at 0° C., sodium dihydrogen orthophosphate (174 mg) dissolved in water (1 mL) was added followed by aqueous H₂O₂ (30% w/v, 0.45 mL) and sodium chlorite (102 mg). After 4 h, the reaction mixture was quenched by aqueous 10% Na₂SO₃ solution and the reaction mixture was extracted with ethyl acetate, washed with water, brine and dried (Na₂SO₄) and concentrated. Purification by column chromatography afforded compound 1 (R=Boc, R¹═OH, R²=Me with 6S stereochemistry) (200 mg, 98% yield) as colorless oil. Data for 1 (R=Boc, R¹═OH, R²=Me with 6S stereochemistry): R_{f}=0.45 (silica, 1:9 MeOH/CHCl₃ with 1% AcOH); [α]_{D}²³=−52.8 (c 1.14, MeOH); ¹H NMR (200 MHz, CDCl₃) δ 7.17 (br d, J=2.2 Hz, 1H, aromatic), 6.29 (d, J=2.2 Hz, 1H, aromatic), 5.04 (br m, 1H, NH), 4.93 (br m, 1H, CHNH), 1.48 (d, J=6.59 Hz, 3H, CH3), 1.42 (s, 9H, t-butyl).

### EXAMPLE 2

### Process for Preparing Chiral Furan Amino Acid 1 Wherein C6 Stereochemistry is S and the Substitutions are R=Boc, R¹═OH, R²═CHMe₂

Step 1: Preparation of the Propargyl Alcohol Adduct 4 (R=Boc, R²=CHMe₂ with 6S Stereochemistry)

To a stirred solution of the dibromo compound 3 (6.27 g) in THF (90 mL) at −78° C., nBuLi (1.6 M in hexane, 26 mL) was slowly added. Stirring was continued at −78° C. for 30 minutes and then at room temperature for another 30 minutes. Reaction mixture was recooled to −78° C. and the aldehyde N-Boc-L-valinal (2: R=Boc, R²=CHMe₂ with 6S stereochemistry) (4.41 g), dissolved in THF (20 mL), was added. After 30 minutes, the reaction mixture was quenched with saturated aqueous NH₄Cl solution. The organic layer was separated and the aqueous layer was extracted with EtOAc. The combined organic extracts was washed with brine and dried over anhydrous Na₂SO₄ and filtered. The solvents were removed in rotary evaporator and the crude mixture was purified using flash column chromatography (SiO₂, 16-20% EtOAc in petroleum ether eluant) to afford the propargyl alcohol adduct 4 (R=Boc, R²=CHMe₂ with 6S stereochemistry) (4.06 g) as oil in 63% yield. Data for 4 (R=Boc, R²=CHMe₂ with 6S stereochemistry): R_{f}=0.5 (silica, 40% EtOAc/Hexane); ¹H NMR (300 MHz, CDCl₃) δ 4.7 (m, 1H, CHOH), 4.59 (d, J=9.07 Hz, 1H, NH), 4.12 (m, 1H, CHCH₂), 3.88 (m, 2H, CH₂), 3.54 (m, 1H, CHNH), 1.78 (m, 1H, CH(CH₃)₂), 1.46 (s, 9H, t-butyl), 1.45 (s, 6H, acetonide protons), 0.99 (d, J=6.8 Hz, 6H, CH₃).

Step 2: Preparation of the Cis-Allylic Alcohol Intermediate 5 (R=Boc, R²=CHMe₂ with 6S Stereochemistry)

Nickel acetate tetrahydrate (2.91 g) was dissolved in 95% ethanol (129 mL) and placed under H₂. A solution of NaBH₄ in absolute ethanol (1 M, 11.7 mL) was added to the reaction mixture under vigorous stirring at room temperature, followed after 30 minutes by ethylene diamine (3.13 mL) and compound 4 (R=Boc, R²=CHMe₂ with 6S stereochemistry) (3.83 g) dissolved in ethanol (15 mL). The reaction progress was monitored by TLC checking. After 1 h, reaction mixture was poured into large excess of hexane and filtered through short Celite pad and the filter cake was washed with diethyl ether. The combined organic extracts were washed with 1N HCl, water and brine, dried (Na₂SO₄), filtered and concentrated in vacuo. Flash chromatography (SiO₂, 18-24% EtOAc in petroleum ether eluant) of the residue afforded cis-allylic alcohol intermediate 5 (R=Boc, R²=CHMe₂ with 6S stereochemistry) (2.31 g, 60% yield) as colorless oil. Data for 5 (R=Boc, R²=CHMe₂ with 6S stereochemistry): R_{f}=0.45 (silica, 30% EtOAc/Hexane); ¹H NMR (300 MHz, CDCl₃) δ 5.65 (m, 1H, olefinic proton), 5.54 (m, 1H, olefinic proton), 4.71 (bs, 1H, NH), 4.5 (m, 1H, CHOH), 4.09 (m, 1H, CH), 3.55 (m, 2H, CH₂), 3.24 (m, 1H, CHNH), 1.94 (m, 1H, CH(CH₃)₂), 1.44 (s, 9H, t-butyl), 1.43 (s, 6H, acetonide methyls), 1.0 (d, J=6.8 Hz, 3H, CH₃), 0.93 (d, J=6.8 Hz, 3H, CH₃).

Steps 34: Preparation of the “cis-2-butene-1,4-diol” Intermediate 6 (R=Boc, R²=CHMe₂ with 6S Stereochemistry)

A solution of compound 5 (R=Boc, R²=CHMe₂ with 6S stereochemistry) (2.18 g) in methanol (35 mL) was treated with CSA (1.54 g) at 0° C. After 4 h, the reaction was quenched by adding saturated aqueous NaHCO₃ solution (till pH 8) and extracted with ethyl acetate. The combined organic extracts were washed with brine, dried (Na₂SO₄), filtered and concentrated in vacuo. The crude mixture was purified by flash chromatography (SiO₂, 4-6% MeOH in CHCl₃ eluant) to afford the Z-triol (1.33 g, 70% yield).

To the stirred solution of the triol (1 g) in CH₂Cl₂ (20 mL) at −78° C. were added 2,4,6-collidine (1 mL) followed by acetyl chloride (0.3 mL). After 10 h, it was quenched by adding saturated aqueous NH₄Cl solution, extracted with ethyl acetate, washed with 1N HCl, water, brine, dried (Na₂SO₄), filtered and concentrated in vacuo. Column chromatography (SiO₂, 3-5% MeOH in CHCl₃ eluant) of the residue afforded pure mono acetylated “cis-2-butene-1,4-diol” intermediate 6 (R=Boc, R²=CHMe₂ with 6S stereochemistry) (928 mg, 80%) as colorless oil. Data for 6 (R=Boc, R²=CHMe₂ with 6S stereochemistry): R_{f}=0.45 (silica, 10% MeOH/CHCl₃); ¹H NMR (300 MHz, CDCl₃) δ 5.66 (dd, J=11.33, 7.93 Hz, 1H, olefinic proton), 5.54 (dd, J=11.33, 8.31 Hz, 1H, olefinic proton), 4.72-4.67 (m, 1H, CHOH), 4.4 (dd, J=7.93, 6.8 Hz, 1H, CH), 4.18 (dd, J=11.33, 3.4 Hz, 1H one of the CH₂), 3.93 (dd, J=11.33, 7.55 Hz, 1H, one of the CH₂), 2.1 (s, 3H, COCH₃), 2 (m, 1H, CH(CH₃)₂), 1.42 (s, 9H, t-butyl), 0.97 (d, J=6.8 Hz, 3H, CH₃), 0.92 (d, J=6.8 Hz, 3H, CH₃).

Steps 5-6: Preparation of the Chiral Furanyl Alcohol Intermediate 7 (R=Boc, R²=CHMe₂ with 6S Stereochemistry)

To a stirred solution of compound 6 (R=Boc, R²=CHMe₂ with 6S stereochemistry) (0.9 g) in CH₂Cl₂ (32 mL), pyridinium chlorochromate (1.012 g) was added. After 30 minutes, the reaction mixture was diluted with excess diethyl ether and filtered through a short celite pad and the filter cake was washed with diethyl ether. The combined organic extracts were washed with 1N HCl, water, brine, dried (Na₂SO₄), filtered and concentrated in vacuo. The residual oil was purified by column chromatography (SiO₂, 10% EtOAc in petroleum ether eluant) to give pure 2,5-disubstituted furan derivative (422 mg, 50%) as colorless oil.

The resulting compound (0.3 g) was dissolved in methanol (4 mL), cooled to 0° C., and then anhydrous potassium carbonate (0.2 g) was added. The reaction mixture was stirred at the same temperature for 15 minutes. It was diluted with ethyl acetate and washed with water, brine, dried (Na₂SO₄), filtered and concentrated in vacuo. Purification by column chromatography (SiO₂, 20% EtOAc in petroleum ether eluant) afforded the chiral furanyl alcohol intermediate 7 (R=Boc, R²=CHMe₂ with 6S stereochemistry) (245 mg, 95% yield) as colorless oil. Data for 7 (R=Boc, R²=CHMe₂ with 6S stereochemistry): R_{f}=0.5 (silica, 30% EtOAc/Hexane); [α]_{D}²³=−59.9 (c 1.76, CHCl₃); ¹H NMR (300 MHz, CDCl₃) δ 6.16 (d, J=2.93 Hz, 1H, one of the furan ring protons), 6.06 (d, J=2.93 Hz, 1H, one of the furan ring protons), 4.84 (d, J=8.79 Hz, 1H, NH), 4.53 (s, 2H, CH₂OH), 4.52 (m, 1H, CHNH) 2.09 (m, 1H, CH(CH₃)₂), 1.44 (s, 9H, t-butyl), 0.94 (d, J=6.59 Hz, 3H, CH₃), 0.88 (d, J=6.59 Hz, 3H, CH₃).

Steps 7-8: Preparation of the Chiral Furan Amino Acid 1 (R=Boc, R¹═OH, R²=CHMe₂ with 6S Stereochemistry)

To a stirred ice-cooled solution of alcohol 7 (R=Boc, R²=CHMe₂ with 6S stereochemistry) (0.2 mg) in dry CH₂Cl₂ (1.6 mL) and dry DMSO (2 mL), Et₃N (0.52 mL) and SO₃-py complex (589 mg) were sequentially added. The reaction mixture was allowed to attain the room temperature slowly and stirred at the same temperature for another 1 h. After 1 h, it was quenched with saturated aqueous NH₄Cl solution, extracted with ether, washed with brine, dried (Na₂SO₄), filtered and concentrated in vacuo. Purification by column chromatography (SiO₂, 17-20% EtOAc in petroleum ether eluant) afforded pure aldehyde (144 mg, 85%) as colorless liquid.

To the stirred solution of the aldehyde (119 mg) in CH₃CN (4 mL) at 0° C., NaH₂PO₄.2H₂O (96.1 mg) dissolved in water (1 mL) was added followed by aqueous H₂O₂ (0.25 mL, 30% w/v) and sodium chlorite (56 mg). After 4 h, the reaction mixture was quenched by aqueous 10% Na₂SO₃ solution (2 mL) at 0° C. and the reaction mixture was extracted with ethyl acetate, washed with water, brine, dried (Na₂SO₄), filtered and concentrated in vacuo. Purification by column chromatography (SiO₂, 7-10% MeOH in Chloroform eluant) afforded compound 1 (R=Boc, R¹═OH, R²=CHMe₂ with 6S stereochemistry) (110 mg, 88% yield) as white solid. Data for 1 (R=Boc, R¹═OH, R²=CHMe₂ with 6S stereochemistry): R_{f}=0.5 (silica, 1:9 MeOH/CHCl₃ with 1% AcOH); ¹H NMR (200 MHz, CDCl₃) δ 7.18 (br 1H, one of the furan ring protons), 6.39 (br, 1H, one of the furan ring protons), 5.09 (br, 1H, NH), 4.61 (br, 1H, CHNH), 2.2 (m, 1H, CH(CH₃)₂), 1.42 (s, 9H, t-butyl), 0.95 (d, J=6.69 Hz, 3H, CH₃), 0.89 (d, J=6.69 Hz, 3H, CH₃).

### EXAMPLE 3

### Process for Preparing Chiral Furan Amino Acid 1 Wherein C6 Stereochemistry is S and the Substitutions are R=Boc, R¹═OH, R²=CH₂Ph

Step 1: Preparation of the Propargyl Alcohol Adduct 4 (R=Boc, R²=CH₂Ph with 6S Stereochemistry)

To a stirred solution of the dibromo compound 3 (7.82 g) in THF (90 mL) at −78° C., nBuLi (1.6M in hexane, 32.5 mL) was slowly added. Stirring was continued at −78° C. for 30 minutes and then at room temperature for another 30 minutes. Reaction mixture was recooled to −78° C. and the aldehyde N-Boc-L-phenylalaninal (2: R=Boc, R²=CH₂Ph with 6S stereochemistry) (5.45 g), dissolved in THF (20 mL), was added. After 30 minutes, the reaction mixture was quenched with saturated aqueous NH₄Cl solution. The organic layer was separated and the aqueous layer was extracted with EtOAc. The combined organic extracts was washed with brine and dried over anhydrous Na₂SO₄ and filtered. The solvents were removed in rotary evaporator and the crude mixture was purified using flash column chromatography (SiO₂, 20-25% EtOAc in petroleum ether eluant) to afford the propargyl alcohol adduct 4 (R=Boc, R²=CH₂Ph with 6S stereochemistry) (5.34 g, 65%) as yellow solid. Data for 4 (R=Boc, R²=CH₂Ph with 6S stereochemistry): R_{f}=0.45 (silica, 40% EtOAc/Hexane); ¹H NMR (200 MHz, CDCl₃) δ 7.23 (m, 5H, aromatic protons), 4.82-4.65 (m, 2H, CHOH & NH), 4.37 (br, 1H, CHNH), 4.19-4.06 (m, 2H, CH & one of the CH₂), 3.9 (m, 1H, one of the CH₂), 2.91 (m, 2H, CH₂Ph), 1.39-1.38 (m, 15H, t-butyl & acetonide methyls).

Step 2: Preparation of the Cis-Allylic Alcohol Intermediate 5 (R=Boc, R²=CH₂Ph with 6S Stereochemistry)

Nickel acetate tetrahydrate (2.91 g) was dissolved in 95% ethanol (129 mL) and placed under H₂. A solution of NaBH₄ in absolute ethanol (1 M, 11.7 ml) was added to the reaction mixture under vigorous stirring at room temperature, followed after 30 minutes by ethylene diamine (3.13 mL) and compound 4 (R=Boc, R²=CH₂Ph with 6S stereochemistry) (4.39 g) dissolved in ethanol (15 mL). The reaction progress was monitored by TLC checking. After 1 h, reaction mixture was poured into large excess of hexane and filtered through short Celite pad and the filter cake was washed with diethyl ether. The combined organic extracts were washed with 1N HCl, water, brine, dried (Na₂SO₄), filtered and concentrated in vacuo. Flash chromatography (SiO₂, 22-25% EtOAc in petroleum ether eluant) of the residue afforded cis-allylic alcohol intermediate 5 (R=Boc, R²=CH₂Ph with 6S stereochemistry) (2.87 g, 65% yield) as colorless oil. Data for 5 (R=Boc, R²=CH₂Ph with 6S stereochemistry): R_{f}=0.45 (silica, 40% EtOAc/Hexane); ¹H NMR (200 MHz, CDCl₃) δ 7.21 (m, 5H, aromatic protons), 5.82-5.55 (m, 2H, olefinic protins), 4.78 (m, 1H, NH), 4.62-4.34 (m, 2H, CHOH & CH), 4.06 (m, 1H, CHNH), 3.51 (m, 2H, CH₂), 2.85 (m, 2H, CH₂Ph), 1.39-1.32 (m, 15H, t-butyl & acetonide methyls).

Steps 34: Preparation of the “cis-2-butene-1,4-diol” Intermediate 6 (R=Boc, R²=CH₂Ph with 6S Stereochemistry)

A solution of compound 5 (R=Boc, R²=CH₂Ph with 6S stereochemistry) (2.5 g) in methanol (30 mL) was treated with CSA (1.54 g) at 0° C. After 4 h, the reaction was quenched by adding saturated aqueous NaHCO₃ solution (till pH 8) and extracted with ethyl acetate. The combined organic extracts were washed with brine, dried (Na₂SO₄), filtered and concentrated in vacuo. The crude mixture was purified by flash chromatography (SiO₂, 6-8% MeOH in CHCl₃ eluant) to afford the Z-triol (1.56 g, 70% yield).

To the stirred solution of the triol (1.3 g) in CH₂Cl₂ (20 mL) at −78° C. were added 2,4,6-collidine (1 mL) followed by acetyl chloride (0.3 mL). After 10 h, it was quenched by adding saturated aqueous NH₄Cl solution, extracted with ethyl acetate, washed with 1N HCl, water, brine, dried (Na₂SO₄), filtered and concentrated in vacuo. Column chromatography (SiO₂, 3-5% MeOH in CHCl₃ eluant) of the residue afforded pure mono acetylated “cis-2-butene-1,4-diol” intermediate 6 (R=Boc, R²=CH₂Ph with 6S stereochemistry) (1.32 g, 90%) as colorless oil. Data for 6 (R=Boc, R²=CH₂Ph with 6S stereochemistry): R_{f}=0.45 (silica, 10% MeOH/CHCl₃); ¹H NMR (200 MHz, CDCl₃) δ 7.21 (m, 5H, aromatic protons), 5.68-5.45 (m, 2H, olefinic protons), 4.65 (m, 2H, CHOH & NH), 4.45 (m, 1H, CHOH), 4.05 (m, 2H, CH₂), 3.8 (m, 1H, CHNH), 2.85 (m, 2H, CH₂Ph), 2.04 (s, 3H, COCH₃), 1.25 (m, 15H, t-butyl).

Steps 5-6: Preparation of the Chiral Furanyl Alcohol Intermediate 7 (R=Boc, R²=CH₂Ph with 6S Stereochemistry)

To a stirred solution of compound 6 (R=Boc, R²=CH₂Ph with 6S stereochemistry) (1 g) in CH₂Cl₂ (30 mL), pyridinium chlorochromate (1 g) was added. After 30 minutes, the reaction mixture was diluted with excess diethyl ether and filtered through a short celite pad and the filter cake was washed with diethyl ether. The combined organic extracts were washed with 1N HCl, water, brine, dried (Na₂SO₄), filtered and concentrated in vacuo. The residual oil was purified by column chromatography (SiO₂, 12% EtOAc in petroleum ether eluant) to give pure 2,5-disubstituted furan derivative (455 mg, 48%) as colorless oil.

The resulting compound (300 mg) was dissolved in methanol (5 mL), cooled to 0° C., and then anhydrous potassium carbonate (174 mg) was added. The reaction mixture was stirred at the same temperature for 15 minutes. It was diluted with ethyl acetate and washed with water, brine, dried (Na₂SO₄), filtered and concentrated in vacuo. Purification by column chromatography (SiO₂, 35-40% EtOAc in petroleum ether eluant) afforded the chiral furanyl alcohol intermediate 7 (R=Boc, R²=CH₂Ph with 6S stereochemistry) (256 mg, 96% yield) as colorless oil. Data for 7 (R=Boc, R=CH₂Ph with 6S stereochemistry): R_{f}=0.5 (silica, 40% EtOAc/hexane); ¹H NMR (200 MHz, CDCl₃) δ 7.2 (m, 3H, aromatic protons), 7.02 (m, 2H, aromatic protons), 6.12 (d, J=2.97 Hz, 1H, one of the furan ring protons), 5.93 (d, J=2.97 Hz, 1H, one of the furan ring protons), 4.94 (m, 1H, CHNH), 4.81 (d, J=8.92 Hz, 1H, NH), 4.53 (s, 2H, CH₂OH), 3.09 (d, J=6.69 Hz, 2H, CH₂Ph), 1.39 (s, 9H, t-butyl).

Steps 7-8: Preparation of the Chiral Furan Amino Acid 1 (R=Boc, R¹═OH, R²=CH₂Ph with 6S Stereochemistry)

To a stirred ice-cooled solution of alcohol 7 (R=Boc, R²=CH₂Ph with 6S stereochemistry) (200 mg) in dry CH₂Cl₂ (1.6 mL) and dry DMSO (2 mL), Et₃N (0.44 mL) and SO₃-py complex (501 mg, 3.15 mmol) were sequentially added. The reaction mixture was allowed to attain the room temperature slowly and stirred at the same temperature for another 1 h. After 1 h, it was quenched with saturated aqueous NH₄Cl solution, extracted with ether, washed with brine, dried (Na₂SO₄), filtered and concentrated in vacuo. Purification by column chromatography (SiO₂ 17-20% EtOAc in petroleum ether eluant) afforded pure aldehyde (155 mg, 78%) as colorless liquid. To the stirred solution of the aldehyde (118 mg) in CH₃CN (4 mL) at 0° C., NaH₂PO₄.2H₂O (81 mg) dissolved in water (1 mL) was added followed by aqueous H₂O₂ (0.21 mL, 30% w/v) and sodium chlorite (47 mg). After 4 h, the reaction mixture was quenched by aqueous 10% Na₂SO₃ solution at 0° C. and the reaction mixture was extracted with ethyl acetate, washed with water, brine, dried (Na₂SO₄), filtered and concentrated in vacuo. Purification by column chromatography (SiO₂, 7-10% MeOH in Chloroform eluant) afforded compound 1 (R=Boc, R¹═OH, R²=CH₂Ph with 6S stereochemistry) (115 mg, 92% yield) as white solid. Data for 1 (R=Boc, R¹═OH, R²=CH₂Ph with 6S stereochemistry): R_{f}=0.5 (silica, 10 MeOH/CHCl₃ with 1% AcOH); ¹H NMR (200 MHz, CDCl₃) δ 7.18 (m, 5H, aromatic protons), 7.05 (br, 1H, one of the furan ring protons), 6.12 (br, 1H, one of the furan ring protons), 5.03 (m, 2H, NH & CHNH), 3.16 (m, 2H, CH₂Ph), 1.39 (s, 9H, t-butyl).

### EXAMPLE 4

### Process for Preparing Chiral Furan Amino Acid 1 Wherein C6 Stereochemistry is S and the Substitutions are R=Boc, R¹═OH, R²=Ph

Step 1: Preparation of the Propargyl Alcohol Adduct 4 (R=Boc, R²=Ph with 65 Stereochemistry)

To a stirred solution of the dibromo compound 3 (6.0 g) in THF (80 mL) at −78° C., nBuLi (1.6 M in hexane, 25 mL) was slowly added. Stirring was continued at −78° C. for 30 minutes and then at room temperature for another 30 minutes. Reaction mixture was recooled to −78° C. and the aldehyde N-Boc-L-phenylglycinal (2: R=Boc, R²=Ph with 6S stereochemistry) (3.98 g), dissolved in THF (20 mL), was added. After 30 minutes, the reaction mixture was quenched with saturated aqueous NH₄Cl solution. The organic layer was separated and the aqueous layer was extracted with EtOAc. The combined organic extracts were washed with brine and dried over anhydrous Na₂SO₄ and filtered. The solvents were removed in rotary evaporator and the crude mixture was purified using flash column chromatography (SiO₂, 16-20% EtOAc in petroleum ether eluant) to afford the propargyl alcohol adduct 4 (R=Boc, R²=Ph with 6S stereochemistry) (3.76 g, 62%) as colorless liquid. Data for 4 (R=Boc, R²=Ph with 6S stereochemistry): R_{f}=0.45 (silica, 40% EtOAc/Hexane); ¹H NMR (200 MHz, CDCl₃) δ 7.29 (m, 5H, aromatic protons), 5.27-5.18 (m, 2H, CHOH & NH), 5 (m, 1H, CHNH), 4.94 (m, 1H, CH), 4.03 (m, 2H, CH₂), 1.44 (s, 9H, t-butyl), 1.41 (s, 6H, acetonide methyls).

Step 2: Preparation of the Cis-Allylic Alcohol Intermediate 5 (R=Boc, R²=Ph with 6S Stereochemistry)

Nickel acetate tetrahydrate (2.41 g) was dissolved in 95% ethanol (106 mL) and placed under H₂. A solution of NaBH₄ in absolute ethanol (1 M, 9.7 mL) was added to the reaction mixture under vigorous stirring at room temperature, followed after 30 minutes by ethylene diamine (2.6 mL) and compound 4 (R=Boc, R²=Ph with 6S stereochemistry) (3.5 g) dissolved in ethanol (20 mL). The reaction progress was monitored by TLC checking. After 1 h, reaction mixture was poured into large excess of hexane and filtered through short Celite pad and the filter cake was washed with diethyl ether. The combined organic extract was washed with 1N HCl, water and brine, dried (Na₂SO₄), filtered and concentrated in vacuo. Flash chromatography (SiO₂, 20-22% EtOAc in petroleum ether eluant) of the residue afforded cis-allylic alcohol intermediate 5 (R=Boc, R²=Ph with 6S stereochemistry) (2.46 g, 70% yield) as colorless oil. Data for 5 (R=Boc, R²=Ph with 6S stereochemistry): R_{f}=0.45 (silica, 40% EtOAc/hexane); ¹H NMR (200 MHz, CDCl₃) δ 7.25 (m, 5H, aromatic protons), 5.87-5.55 (m, 2H, olefinic protons), 5.25 (m, 2H, CHOH, NH), 4.99 (m, 1H, CHNH), 4.58 (m, 1H, CH), 3.90 (m, 2H, CH₂), 1.44 (s, 9H, t-butyl), 1.41 (s, 6H, acetonide methyls).

Steps 3-4: Preparation of the “cis-2-butene-1,4-diol” Intermediate 6 (R=Boc, R²=Ph with 6S Stereochemistry)

A solution of compound 5 (R=Boc, R²=Ph with 6S stereochemistry) (2 g) in methanol (30 mL) was treated with CSA (1.28 g) at 0° C. After 4 h, the reaction was quenched by adding saturated aqueous NaHCO₃ solution (till pH 8) and extracted with ethyl acetate. The combined organic extracts were washed with brine, dried (Na₂SO₄), filtered and concentrated in vacuo. The crude mixture was purified by flash chromatography (SiO₂, 6-8% MeOH in CHCl₃ eluant) to afford the Z-triol (1.25 g, 70% yield).

To the stirred solution of the triol (1 g) in CH₂Cl₂ (20 mL) at −78° C. were added 2,4,6-collidine (0.82 mL) followed by acetyl chloride (0.24 mL). After 10 h, it was quenched by adding saturated aqueous NH₄Cl solution, extracted with ethyl acetate, washed with 1N HCl, water, brine, dried (Na₂SO₄), filtered and concentrated in vacuo. Column chromatography (SiO₂, 3-5% MeOH in CHCl₃ eluant) of the residue afforded pure mono acetylated “cis-2-butene-1,4-diol” intermediate 6 (R=Boc, R²=Ph with 6S stereochemistry) (961 mg, 85%) as colorless oil. Data for 6 (R=Boc, R²=Ph with 6S stereochemistry): R_{f}=0.45 (silica, 10% MeOH/CHCl₃); ¹H NMR (200 MHz, CDCl₃) δ 7.29 (m, 5H, aromatic protons), 5.87-5.55 (m, 2H, olefinic protons), 5.25 (m, 2H, CHOH & NH), 4.85 (m, 1H, CHNH), 4.61 (m, 1H, CHOH), 4.21 (m, 2H, CH₂), 2.1 (s, 3H, COCH₃), 1.44 (s, 9H, t-butyl).

Steps 5-6: Preparation of the Chiral Furanyl Alcohol Intermediate 7 (R=Boc, R²=Ph with 6S Stereochemistry)

To a stirred solution of compound 6 (R=Boc, R²=Ph with 6S stereochemistry) (800 mg) in CH₂Cl₂ (25 mL), pyridinium chlorochromate (849 mg) was added. After 30 minutes, the reaction mixture was diluted with excess diethyl ether and filtered through a short celite pad and the filter cake was washed with diethyl ether. The combined organic extracts were washed with 1N HCl, water, brine, dried (Na₂SO₄), filtered and concentrated in vacuo. The residual oil was purified by column chromatography (SiO₂, 12% EtOAc in petroleum ether eluant) to give pure 2,5-disubstituted furan derivative (304 mg, 40%) as colorless oil.

The resulting compound (300 mg) was dissolved in methanol (5 mL), cooled to 0° C., and then anhydrous potassium carbonate (178 mg) was added. The reaction mixture was stirred at the same temperature for 15 minutes. It was diluted with ethyl acetate and washed with water, brine, dried (Na₂SO₄), filtered and concentrated in vacuo. Purification by column chromatography (SiO₂, 35-40% EtOAc in petroleum ether eluant) afforded the chiral furanyl alcohol intermediate 7 (R=Boc, R²=Ph with 6S stereochemistry) (248 mg, 95% yield) as colorless oil. Data for 7 (R=Boc, R²=Ph with 6S stereochemistry): R_{f}=0.45 (silica, 40% EtOAc/Hexane); ¹H NMR (400 MHz, CDCl₃) δ 7.29 (m, 5H, aromatic protons), 6.16 (d, J=3.05 Hz, 1H, one of the furan ring protons), 6.02 (d, J=3.05 Hz, 1H, one of the furan ring protons), 5.87 (br, 1H, NH), 5.25 (d, J=8.52 Hz, 1H, CHNH), 4.51 (s, 2H, CH₂OH), 1.44 (s, 9H, t-butyl).

Steps 7-8: Preparation of the Chiral Furan Amino Acid 1 (R=Boc, R¹═OH, R²=Ph with 6S Stereochemistry)

To a solution of oxalyl chloride (0.09 mL) in dry CH₂Cl₂ (2 mL) at −78° C., DMSO (0.16 mL) was added dropwise with stirring under N₂ atmosphere. After 15 min, the chiral furanyl alcohol intermediate 7 (R=Boc, R²=Ph with 6S stereochemistry) (220 mg) in dry CH₂Cl₂ (1 mL) was added to the reaction mixture. After 30 min of stirring at −78° C., Et₃N (0.5 mL) was added and stirred at the same temperature for another 30 min, finally at the 0° C. for 0.5 h. The reaction mixture was quenched with saturated aqueous NH₄Cl solution and extracted with CH₂Cl₂. The combined organic extracts were washed with brine, dried (Na₂SO₄), filtered and concentrated in vacuo. Purification by column chromatography (SiO₂ 15-20% EtOAc in petroleum ether eluant) afforded pure aldehyde (162 mg, 75%) as colorless liquid.

To the stirred solution of the aldehyde (108 mg) in CH₃CN (4 mL) at 0° C., NaH₂PO₄.2H₂O (79 mg) dissolved in water (1 mL) was added followed by aqueous H₂O₂ (0.2 mL, 30% w/v) and sodium chlorite (46 mg). After 4 h, the reaction mixture was quenched by aqueous 10% Na₂SO₃ solution at 0° C. and the reaction mixture was extracted with ethyl acetate, washed with water, brine, dried (Na₂SO₄), filtered and concentrated in vacuo. Purification by column chromatography (SiO₂, 7-10% MeOH in CHCl₃ eluant) afforded afforded compound 1 (R=Boc, R¹═OH, R²=Ph with 6S stereochemistry) (102 mg, 90% yield) as white solid. Data for 1 (R=Boc, R¹═OH, R²=Ph with 6S stereochemistry): R_{f}=0.5 (silica, 10% MeOH/CHCl₃ with 1% AcOH); ¹H NMR (200 MHz, CDCl₃) δ 7.29 (m, 5H, aromatic protons), 7.15 (br, 1H, one of the furan ring protons), 6.21 (br, 1H, one of the furan ring protons), 5.85 (br, 1H, CHNH), 5.43 (br, 1H, NH), 1.44 (s, 9H, t-butyl).

1. An unnatural chiral furan amino acids carrying natural amino acid side-chains at C6-position and having a general structure 1 as shown in Formula I 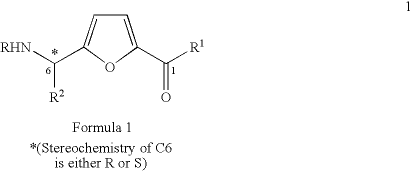
Wherein;
R═H, tert-butoxycarbonyl (Boc), benzyloxycarbonyl (Cbz), 9-fluorenylmethyl (Fmoc), acetyl or salts such as HCl, CF₃COOH.H and others;
R¹=—OH, —O-alkyl, —O-arylalkyl, -amine, -alkylamine, -arylalkylamine, and others;
R²=CH₃—, (CH₃)₂CH—, (CH₃)₂CHCH₂—, CH₃CH₂CH(CH₃)—, alkyl groups;
(OR³)CH₂—, CH₃(OR³)CH—, (R³S)CH₂—, CH₃SCH₂CH₂—, (RHN)CH₂CH₂CH₂CH₂—; (CONH₂)CH₂—, (CONH₂)CH₂CH₂—, (CO₂R⁴)CH₂—, (CO₂R⁴)CH₂CH₂—, Ph-, Ar—; PhCH₂—, ArCH₂—, Phenylalkyl-, arylalkyl-, (indolyl)CH₂—, (imidazolyl)CH₂—, and all other amino acid side-chains;
R³═H, tert-butyl, alkyl, benzyl, arylCH₂, CO(alkyl), CO(arylalkyl), SO₃H, PO₃H₂, silyl and others;
R⁴═H, tert-butyl, alkyl, benzyl, arylCH₂, and others;
R—R═—(CH₂)ₙ— (n=2, 3, 4 . . . ).

2. A chiral furan amino acid as claimed in claim 1, wherein if the stereochemistry of C6 is S and the substitutions are R¹=Me, R²=Me and R=Boc having a structural formula 2 shown here below

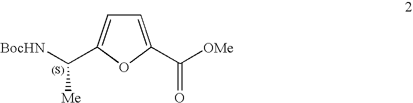

3. A chiral furan amino acid as claimed in claim 1, wherein if the stereochemistry of C6 is S and the substitutions are R¹═OH, R²=Me and R=Boc having a structural formula 3 shown here below

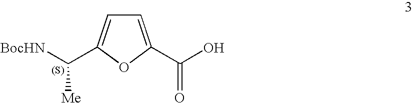

4. A chiral furan amino acid as claimed in claim 1, wherein if the stereochemistry of C6 is S and the substitutions are R¹=OMe, R²=Me and R═CF₃COOH.H having a structural formula 4 shown here below

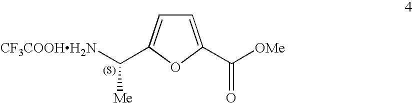

5. A chiral furan amino acid as claimed in claim 1, wherein if the stereochemistry of C6 is S and the substitutions are R¹═OH, R²=Me and R═CF₃COOH.H having a structural formula 5 shown here below

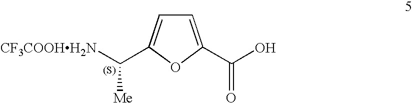

6. A chiral furan amino acid as claimed in claim 1, wherein if the stereochemistry of C6 is R and the substitutions are R¹=OMe, R²=Me and R=Boc having a structural formula 6 shown here below

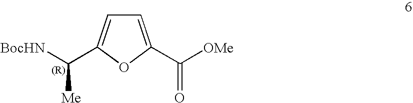

7. A chiral furan amino acid as claimed in claim 1, wherein if the stereochemistry of C6 is R and the substitutions are R¹═OH, R²=Me and R=Boc having a structural formula 7 shown here below

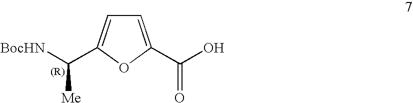

8. A chiral furan amino acid as claimed in claim 1, wherein if the stereochemistry of C6 is R and the substitutions are R¹=OMe, R²=Me and R═CF₃COOH.H having a structural formula 8 shown here below

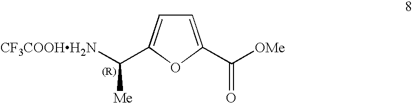

9. A chiral furan amino acid as claimed in claim 1, wherein if the stereochemistry of C6 is R and the substitutions are R¹═OH, R²=Me and R═CF₃COOH.H having a structural formula 9 shown here below

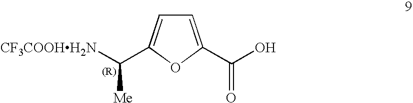

10. A chiral furan amino acid as claimed in claim 1, wherein if the stereochemistry of C6 is S and the substitutions are R¹=OMe, R²=CHMe₂ and R=Boc having a structural formula 10 shown here below

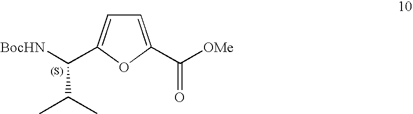

11. A chiral furan amino acid as claimed in claim 1, wherein if the stereochemistry of C6 is S and the substitutions are R¹═OH, R²=CHMe₂ and R=Boc having a structural formula 11 shown here below

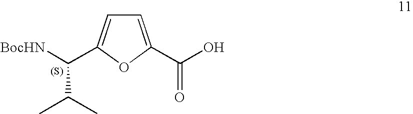

12. A chiral furan amino acid as claimed in claim 1, wherein if the stereochemistry of C6 is S and the substitutions are R¹=OMe, R²=CHMe₂ and R═CF₃COOH.H having a structural formula 12 shown here below

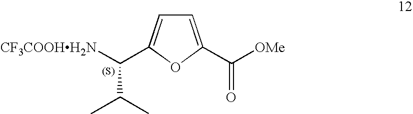

13. A chiral furan amino acid as claimed in claim 1, wherein if the stereochemistry of C6 is S and the substitutions are R¹═OH, R²=CHMe₂ and R═CF₃COOH.H having a structural formula 13 shown here below

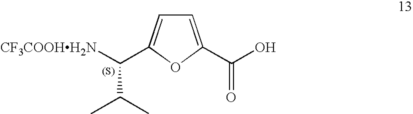

14. A chiral furan amino acid as claimed in claim 1, wherein if the stereochemistry of C6 is R and the substitutions are R¹=OMe, R²=CHMe₂ and R=Boc having a structural formula 14 shown here below

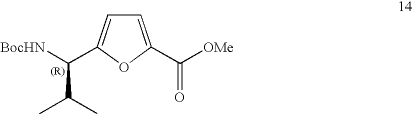

15. A chiral furan amino acid as claimed in claim 1, wherein if the stereochemistry of C6 is R and the substitutions are R¹═OH, R²=CHMe₂ and R=Boc having a structural formula 15 shown here below

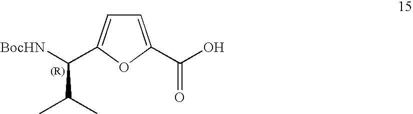

16. A chiral furan amino acid as claimed in claim 1, wherein if the stereochemistry of C6 is R and the substitutions are R¹=OMe, R²=CHMe₂ and R═CF₃COOH.H having a structural formula 16 shown here below

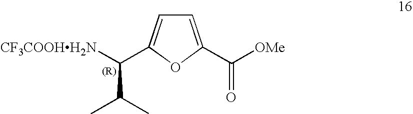

17. A chiral furan amino acid as claimed in claim 1, wherein if the stereochemistry of C6 is R and the substitutions are R¹═OH, R²=CHMe₂ and R═CF₃COOH.H having a structural formula 17 shown here below

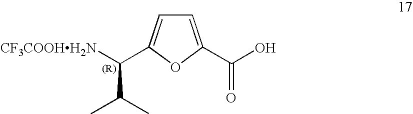

18. A chiral furan amino acid as claimed in claim 1, wherein if the stereochemistry of C6 is S and the substitutions are R¹=OMe, R²=CH₂Ph and R=Boc having a structural formula 18 shown here below

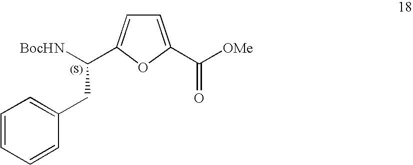

19. A chiral furan amino acid as claimed in claim 1, wherein if the stereochemistry of C6 is S and the substitutions are R¹═OH, R²=CH₂Ph and R=Boc having a structural formula 19 shown here below

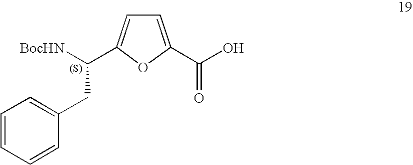

20. A chiral furan amino acid as claimed in claim 1, wherein if the stereochemistry of C6 is S and the substitutions are R¹=OMe, R²=CH₂Ph and R═CF₃COOH.H having a structural formula 20 shown here below

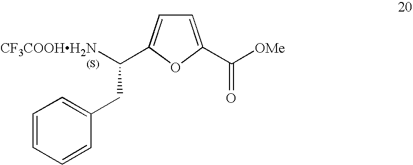

21. A chiral furan amino acid as claimed in claim 1, wherein if the stereochemistry of C6 is S and the substitutions are R¹═OH, R²=CH₂Ph and R═CF₃COOH.H having a structural formula 21 shown here below

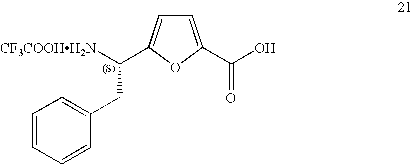

22. A chiral furan amino acid as claimed in claim 1, wherein if the stereochemistry of C6 is R and the substitutions are R¹=OMe, R²=CH₂Ph and R=Boc having a structural formula 22 shown here below

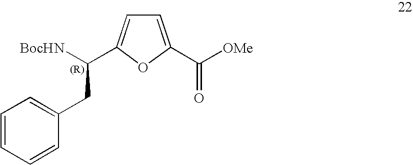

23. A chiral furan amino acid as claimed in claim 1, wherein if the stereochemistry of C6 is R and the substitutions are R¹═OH, R²=CH₂Ph and R=Boc having a structural formula 23 shown here below

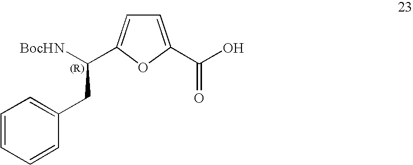

24. A chiral furan amino acid as claimed in claim 1, wherein if the stereochemistry of C6 is R and the substitutions are R¹=OMe, R²=CH₂Ph and R═CF₃COOH.H having a structural formula 24 shown here below

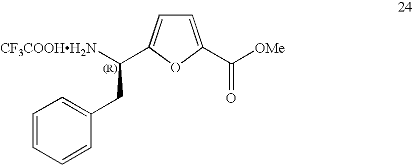

25. A chiral furan amino acid as claimed in claim 1, wherein if the stereochemistry of C6 is R and the substitutions are R¹═OH, R²=CH₂Ph and R═CF₃COOH.Hc having a structural formula 25 shown here below

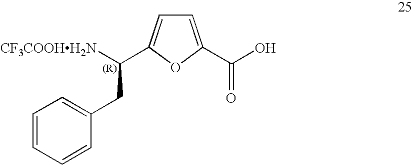

26. A process as claimed in claim 1, wherein if structure 1 with substitution R=Boc, R¹═OH, R²=Me and 6S stereochemistry, has the following characteristics: R_{f}=0.45 (silica, 1:9 MeOH/CHCl₃ with 1% AcOH); [α]_{D}²³=−52.8 (c 1.14, MeOH); ¹H NMR (200 MHz, CDCl₃) δ 7.17 (br d, J=2.2 Hz, 1H, aromatic), 6.29 (d, J=2.2 Hz, 1H, aromatic), 5.04 (br m, 1H, NH), 4.93 (br m, 1H, CHNH), 1.48 (d, J=6.59 Hz, 3H, CH3), 1.42 (s, 9H, t-butyl) and yield up to 98%.

27. A process as claimed in claim 1, wherein if structure 1 with substitution R=Boc, R¹═OH, R²=CHMe₂ and 6S stereochemistry, has the following characteristics: R_{f}=0.5 (silica, 1:9 MeOH/CHCl₃ with 1% AcOH); ¹H NMR (200 MHz, CDCl₃) δ 7.18 (br 1H, one of the furan ring protons), 6.39 (br, 1H, one of the furan ring protons), 5.09 (br, 1H, NH), 4.61 (br, 1H, CHNH), 2.2 (m, 1H, CH(CH₃)₂), 1.42 (s, 9H, t-butyl), 0.95 (d, J=6.69 Hz, 3H, CH₃), 0.89 (d, J=6.69 Hz, 3H, CH₃) and yield up to 88%.

28. A process as claimed in claim 1, wherein if structure 1 with substitution R=Boc, R¹═OH, R²=CH₂Ph and 6S stereochemistry, has the following characteristics: R_{f}=0.5 (silica, 10 MeOH/CHCl₃ with 1% AcOH); ¹H NMR (200 MHz, CDCl₃) δ 7.18 (m, 5H, aromatic protons), 7.05 (br, 1H, one of the furan ring protons), 6.12 (br, 1H, one of the furan ring protons), 5.03 (m, 2H, NH & CHNH), 3.16 (m, 2H, CH₂Ph), 1.39 (s, 9H, t-butyl) and yield up to 92%.

29. A process as claimed in claim 1, wherein if structure 1 with substitution R=Boc, R¹═OH, R²=Ph and 6S stereochemistry, has the following characteristics: R_{f}=0.5 (silica, 10% MeOH/CHCl₃ with 1% AcOH); ¹H NMR (200 MHz, CDCl₃) δ 7.29 (m, 5H, aromatic protons), 7.15 (br, 1H, one of the furan ring protons), 6.21 (br, 1H, one of the furan ring protons), 5.85 (br, 1H, CHNH), 5.43 (br, 1H, NH), 1.44 (s, 9H, t-butyl) and yield up to 90%.

30. A chiral furan amino acids as claimed in claims 5, 9, 13, 17, 21 or 25, wherein N-Fmoc-protected furan amino acid is obtained by treatment with FmocOSu in dioxane-water in the ration of 1:1.

31. A process for preparing unnatural chiral furan amino acids carrying natural amino acid side-chains in C6-position and having a general structure as shown in structure 1 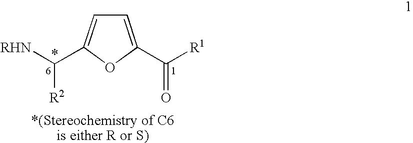
Wherein; R═H, Boc, Cbz, Fmoc, acetyl or salts such as HCl.H, CF₃COOH.H and others;
R¹═—OH, —O-alkyl, —O-arylalkyl, -amine, -alkylamine, -arylalkylamine, and others;
R²═CH₃—, (CH₃)₂CH—, (CH₃)₂CHCH₂—, CH₃CH₂CH(CH₃)—, alkyl groups;
(OR³)CH₂—, CH₃(OR³)CH—, (R³S)CH₂—, CH₃SCH₂CH₂—, (RHN)CH₂CH₂CH₂CH₂—; (CONH₂)CH₂—, (CONH₂)CH₂CH₂—, (CO₂R⁴)CH₂—, (CO₂R⁴)CH₂CH₂—, Ph-, Ar—; PhCH₂—, ArCH₂—, Phenylalkyl-, arylalkyl-, (indolyl)CH₂—, (imidazolyl)CH₂—, and all other amino acid side-chains;
R³═H, tert-butyl, alkyl, benzyl, arylCH₂, CO(alkyl), CO(arylalkyl), SO₃H, PO₃H₂, silyl and others;
R⁴═H, tert-butyl, alkyl, benzyl, arylCH₂, and others;
R—R²=—(CH₂)ₙ— (n=2, 3, 4 . . . );
said process comprising the steps of:
a) addition of Li-acetylide, prepared in-situ by reacting 3,4-O-isopropylidene-1,1-dibromobut-1-en-3,4-diol 3 with n-BuLi, to the chiral N-protected amino aldehyde 2 to obtain the propargyl alcohol adduct 4 as a mixture of isomers having the structure 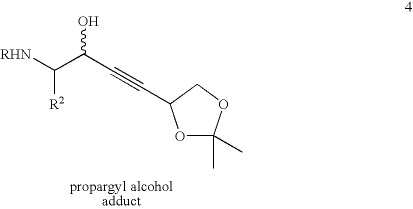
b) selective hydrogenation of the acetylenic moiety to a cis double bond using P2-Ni to get the cis-allylic alcohol intermediate 5 having the structure 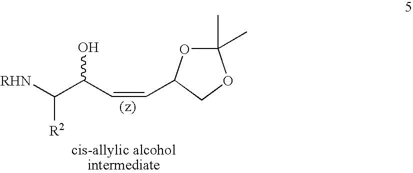
c) treating 5 with acid to deprotect the acetonide and to furnish an intermediate triol
d) selective acylation of the primary hydroxyl group of the triol from of step (c) to obtain the “cis-2-butene-1,4-diol” intermediate 6 having the structure 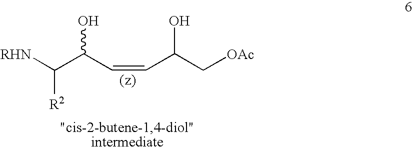
e) oxidation of the “cis-2-butene-1,4-diol” intermediate 6 using pyridinium chlorochromate (PCC) to construct the furan ring
f) deprotection of the intermediate acetate from step (e) in presence of anhydrous K₂CO₃ to obtain the chiral furanyl alcohol intermediate 7 having the structure 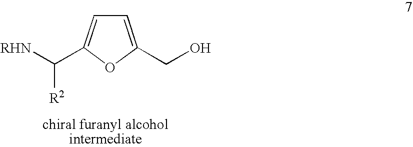
g) oxidation of the primary hydroxyl of the chiral furanyl alcohol intermediate 7 using Swern oxidation process or SO₃-py complex to obtain an aldehyde
h) oxidation of the aldehyde intermediate from step (g) using NaClO₂—H₂O₂ to obtain the desired acid 1 (R¹═OH) having the structure 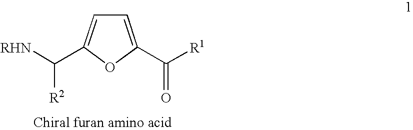
i) transformation of the acid from step (h) into (a) an ester (i) on treatment with CH₂N₂ in ether (1: R¹═OMe), or (ii) an alcohol in the presence of acid (1: R¹═O-alkyl etc.); (b) an amide on treatment with an amine in presence of DCC and HOBt (1: R¹=-amine, -alkylamine, -arylalkylamine).

32. A process as claimed in claim 31 wherein in step (a), if the structure 4 with substitution R=Boc, R²=Me and 6S stereochemistry, has the following characteristics: R_{f}=0.5 (silica, 2:3 ethyl acetate/hexane); ¹H NMR (300 MHz, CDCl₃) δ 4.73-4.68 (ddd, J=6.04, 3.78, 1.51 Hz, 1H, CHOH), 4.65-4.62 (d, J=8.31 Hz, 1H, NH), 4.36-4.32 (ddd, J=6.79, 5.29, 1.51 Hz, 1H, CHCH₂), 4.15-4.09 (dd, J=6.79, 6.04 Hz, 1H, one of the CH₂ protons), 3.91-3.86 (dd, J=6.04, 5.29 Hz, 1H, one of the CH₂ protons), 3.83-3.76 (m, 1H, CHNH), 2.89 (bs, 1H, OH), 1.45 (s, 3H, acetonide methyl protons), 1.442 (s, 9H, t-butyl protons), 1.354 (s, 3H, acetonide methyl protons), 1.247-1.225 (d, J=6.79 Hz, 3H, CH₃) and yield up to 60%.

33. A process as claimed in claim 31 wherein in step (a), if the structure 4 with substitution R=Boc, R²=CHMe₂ and 6S stereochemistry, has the following characteristics: R_{f}=0.5 (silica, 40% EtOAc/Hexane); ¹H NMR (300 MHz, CDCl₃) δ 4.7 (m, 1H, CHOH), 4.59 (d, J=9.07 Hz, 1H, NH), 4.12 (m, 1H, CHCH₂), 3.88 (m, 2H, CH₂), 3.54 (m, 1H, CHNH), 1.78 (m, 1H, CH(CH₃)₂), 1.46 (s, 9H, t-butyl), 1.45 (s, 6H, acetonide protons), 0.99 (d, J=6.8 Hz, 6H, CH₃) and yield up to 63%.

34. A process as claimed in claim 31 wherein in step (a), if the structure 4 with substitution R=Boc, R²=CH₂Ph and 6S stereochemistry, has the following characteristics: R_{f}=0.45 (silica, 40% EtOAc/Hexane); ¹H NMR (200 MHz, CDCl₃) δ 7.23 (m, 5H, aromatic protons), 4.82-4.65 (m, 2H, CHOH & NH), 4.37 (br, 1H, CHNH), 4.19-4.06 (m, 2H, CH & one of the CH₂), 3.9 (m, 1H, one of the CH₂), 2.91 (m, 2H, CH₂Ph), 1.39-1.38 (m, 15H, t-butyl & acetonide methyls) and yield up to 65%.

35. A process as claimed in claim 31 wherein in step (a), if the structure 4 with substitution R=Boc, R²=Ph and 6S stereochemistry, has the following characteristics: R_{f}=0.45 (silica, 40% EtOAc/Hexane); ¹H NMR (200 MHz, CDCl₃) δ 7.29 (m, 5H, aromatic protons), 5.27-5.18 (m, 2H, CHOH & NH), 5 (m, 1H, CHNH), 4.94 (m, 1H, CH), 4.03 (m, 2H, CH₂), 1.44 (s, 9H, t-butyl), 1.41 (s, 6H, acetonide methyls) and yield up to 62%.

36. A process as claimed in claim 31 wherein in step (b), if the structure 5 with substitution R=Boc, R²=Me and 6S stereochemistry, has the following characteristics: R_{f}=0.45 (silica, 2:3 ethyl acetate/hexane); ¹H NMR (200 MHz, CDCl₃) δ 5.62-5.55 (m, 2H, olefinic protons), 4.92-4.68 (m, 2H, CHOH), 4.36-4.27 (bs, 1H, NH), 4.15-4.05 (m, 2H, CH₂OH), 3.71-3.61 (m, 0.1H, CH), 3.06 (bs, 1H, OH), 1.44 (s, 9H, t-butyl protons), 1.40 (s, 3H, acetonide methyl protons), 1.36 (s, 3H, acetonide methyl protons), 1.18-1.15 (d, J=6.69 Hz, 3H, methyl protons) and yield up to 70%.

37. A process as claimed in claim 31 wherein in step (b), if the structure 5 with substitution R=Boc, R²=CHMe₂ and 6S stereochemistry, has the following characteristics: R_{f}=0.45 (silica, 30% EtOAc/Hexane); ¹H NMR (300 MHz, CDCl₃) δ 5.65 (m, 1H, olefinic proton), 5.54 (m, 1H, olefinic proton), 4.71 (bs, 1H, NH), 4.5 (m, 1H, CHOH), 4.09 (m, 1H, CH), 3.55 (m, 2H, CH₂), 3.24 (m, 1H, CHNH), 1.94 (m, 1H, CH(CH₃)₂), 1.44 (s, 9H, t-butyl), 1.43 (s, 6H, acetonide methyls), 1.0 (d, J=6.8 Hz, 3H, CH₃), 0.93 (d, J=6.8 Hz, 3H, CH₃) and yield up to 60%.

38. A process as claimed in claim 31 wherein in step (b) if the structure 5 with substitution R=Boc, R²=CH₂Ph and 6S stereochemistry, has the following characteristics: R_{f}=0.45 (silica, 40% EtOAc/Hexane); ¹H NMR (200 MHz, CDCl₃) δ 7.21 (m, 5H, aromatic protons), 5.82-5.55 (m, 2H, olefinic protins), 4.78 (m, 1H, NH), 4.62-4.34 (m, 2H, CHOH & CH), 4.06 (m, 1H, CHNH), 3.51 (m, 2H, CH₂), 2.85 (m, 2H, CH₂Ph), 1.39-1.32 (m, 15H, t-butyl & acetonide methyls) and yield up to 65%.

39. A process as claimed in claim 31 wherein in step (b), if the structure 5 with substitution R=Boc, R²=Ph and 6S stereochemistry, has the following characteristics: R_{f}=0.45 (silica, 40% EtOAc/hexane); ¹H NMR (200 MHz, CDCl₃) δ 7.25 (m, 5H, aromatic protons), 5.87-5.55 (m, 2H, olefinic protons), 5.25 (m, 2H, CHOH, NH), 4.99 (m, 1H, CHNH), 4.58 (m, 1H, CH), 3.90 (m, 2H, CH₂), 1.44 (s, 9H, t-butyl), 1.41 (s, 6H, acetonide methyls) and yield up to 70%.

40. A process as claimed in claim 31 wherein in step (d), if the structure 6 with substitution R=Boc, R²=Me and 6S stereochemistry, has the following characteristics: R_{f}=0.6 (silica, 1:9 methanol/chloroform); ¹H NMR (200 MHz, CDCl₃) δ 5.66-5.46 (two dd, J=11.89, 6.69 Hz, 2H, olefinic protons), 4.90-4.85 (d, J=8.92 Hz, 1H, NH), 4.66-4.59 (dt, J=6.69, 4.46 Hz, 1H, CHOH), 4.41-4.36 (ddd, J=6.69, 5.02, 4.46 Hz, 1H, CHOH), 4.16-3.98 (two dd, J=11.15, 6.69 and 11.15, 4.46 Hz, 2H, CH₂OAc), 2.09 (s, 3H, CH₃CO), 1.44 (s, 9H, t-butyl), 1.20-1.17 (d, J=6.69 Hz, 3H, CH₃) and yield up to 93%.

41. A process as claimed in claim 31 wherein in step (d), if the structure 6 with substitution R=Boc, R²=CHMe₂ and 6S stereochemistry, has the following characteristics: R_{f}=0.45 (silica, 10% MeOH/CHCl₃); ¹H NMR (300 MHz, CDCl₃) δ 5.66 (dd, J=11.33, 7.93 Hz, 1H, olefinic proton), 5.54 (dd, J=11.33, 8.31 Hz, 1H, olefinic proton), 4.72-4.67 (m, 1H, CHOH), 4.4 (dd, J=7.93, 6.8 Hz, 1H, CH), 4.18 (dd, J=11.33, 3.4 Hz, 1H one of the CH₂), 3.93 (dd, J=11.33, 7.55 Hz, 1H, one of the CH₂), 2.1 (s, 3H, COCH₃), 2 (m, 1H, CH(CH₃)₂), 1.42 (s, 9H, t-butyl), 0.97 (d, J=6.8 Hz, 3H, CH₃), 0.92 (d, J=6.8 Hz, 3H, CH₃) and yield up to 80%.

42. A process as claimed in claim 31 wherein in step (d), if the structure 6 with substitution R=Boc, R²=CH₂Ph and 6S stereochemistry, has the following characteristics: R_{f}=0.45 (silica, 10% MeOH/CHCl₃); ¹H NMR (200 MHz, CDCl₃) δ 7.21 (m, 5H, aromatic protons), 5.68-5.45 (m, 2H, olefinic protons), 4.65 (m, 2H, CHOH & NH), 4.45 (m, 1H, CHOH), 4.05 (m, 2H, CH₂), 3.8 (m, 1H, CHNH), 2.85 (m, 2H, CH₂Ph), 2.04 (s, 3H, COCH₃), 1.25 (m, 15H, t-butyl) and yield up to 90%.

43. A process as claimed in claim 31 wherein in step (d), if the structure 6 with substitution R=Boc, R²=Ph and 6S stereochemistry, has the following characteristics: R_{f}=0.45 (silica, 10% MeOH/CHCl₃); ¹H NMR (200 MHz, CDCl₃) δ 7.29 (m, 5H, aromatic protons), 5.87-5.55 (m, 2H, olefinic protons), 5.25 (m, 2H, CHOH & NH), 4.85 (m, 1H, CHNH), 4.61 (m, 1H, CHOH), 4.21 (m, 2H, CH₂), 2.1 (s, 3H, COCH₃), 1.44 (s, 9H, t-butyl) and yield up to 85%.

44. A process as claimed in claim 31 wherein in step (f), if the structure 7 with substitution R=Boc, R²=Me and 6S stereochemistry, has the following characteristics: R_{f}=0.45 (silica, 1:1 ethyl acetate/hexane); [α]_{D}²³=−59.9 (c 1.76, CHCl₃); ¹H NMR (200 MHz, CDCl₃) δ 6.17-6.14 (d, J=2.97 Hz, 1H, one of the ring protons), 6.08-6.04 (d, J=2.97 Hz, 1H, one of the ring protons), 4.86-4.71 (bs, 2H, NH and CH), 4.52 (s, 2H, CH₂OH), 2.14-1.93 (bs, 1H, OH) 1.48-1.43 (s, 12H, t-butyl group and methyl protons) and yield up to 98%.

45. A process as claimed in claim 31 wherein in step (f), if the structure 7 with substitution R=Boc, R²=CHMe₂ and 6S stereochemistry, has the following characteristics: R_{f}=0.5 (silica, 30% EtOAc/Hexane); [α]_{D}²³=−59.9 (c 1.76, CHCl₃); ¹H NMR (300 MHz, CDCl₃) δ 6.16 (d, J=2.93 Hz, 1H, one of the furan ring protons), 6.06 (d, J=2.93 Hz, 1H, one of the furan ring protons), 4.84 (d, J=8.79 Hz, 1H, NH), 4.53 (s, 2H, CH₂OH), 4.52 (m, 1H, CHNH) 2.09 (m, 1H, CH(CH₃)₂), 1.44 (s, 9H, t-butyl), 0.94 (d, J=6.59 Hz, 3H, CH₃), 0.88 (d, J=6.59 Hz, 3H, CH₃) and yield up to 95%.

46. A process as claimed in claim 31 wherein in step (f), if the structure 7 with substitution R=Boc, R²=CH₂Ph and 6S stereochemistry, has the following characteristics: R_{f}=0.5 (silica, 40% EtOAc/hexane); ¹H NMR (200 MHz, CDCl₃) δ 7.2 (m, 3H, aromatic protons), 7.02 (m, 2H, aromatic protons), 6.12 (d, J=2.97 Hz, 1H, one of the furan ring protons), 5.93 (d, J=2.97 Hz, 1H, one of the furan ring protons), 4.94 (m, 1H, CHNH), 4.81 (d, J=8.92 Hz, 1H, NH), 4.53 (s, 2H, CH₂OH), 3.09 (d, J=6.69 Hz, 2H, CH₂Ph), 1.39 (s, 9H, t-butyl) and yield up to 96%.

47. A process as claimed in claim 31 wherein in step (f), if the structure 7 with substitution R=Boc, R²=Ph and 6S stereochemistry, has the following characteristics: R_{f}=0.45 (silica, 40% EtOAc/Hexane); ¹H NMR (400 MHz, CDCl₃) δ 7.29 (m, 5H, aromatic protons), 6.16 (d, J=3.05 Hz, 1H, one of the furan ring protons), 6.02 (d, J=3.05 Hz, 1H, one of the furan ring protons), 5.87 (br, 1H, NH), 5.25 (d, J=8.52 Hz, 1H, CHNH), 4.51 (s, 2H, CH₂OH), 1.44 (s, 9H, t-butyl) and yield up to 95%.
